(19) United States
(12) Patent Application Publication
(10) Pub. No.: US 2022/0146500 A1
(43) Pub. Date: May 12, 2022

(54) COMPOSITIONS AND METHODS FOR ENHANCING MUCOSAL IMMUNITY

(71) Applicant: Yale University, New Haven, CT (US)

(72) Inventor: Raymond Johnson, Branford, CT (US)

(21) Appl. No.: 17/434,251

(22) PCT Filed: Feb. 27, 2020
(86) PCT No.: PCT/US20/20020

§ 371 Date: Aug. 26, 2021

Related U.S. Application Data
(60) Provisional application No. 62/811,149, filed on Feb. 27, 2019.

(51) Int. Cl. G01N 33/50; G01N 33/68; C12Q 1/6883; G01N 33/564

(52) U.S. Cl. CPC G01N 33/5091; G01N 33/6869; G01N 33/6866; C12Q 1/6883; G01N 33/564; C12Q 2600/158; G01N 2333/5437; G01N 2333/5406; G01N 2333/5409; G01N 2333/57; C12Q 2600/112; G01N 2333/55

(57) ABSTRACT

The invention includes compositions comprising a therapeutic agent that decreases the population of pathological CD4g13 T cells, g13Th1 or g13Th2, in a subject and compositions comprising a therapeutic agent that increases the population of protective CD4g13 T cells, g13Th1 or g13Th2, in a subject. The invention also includes methods for treating an inflammatory or autoimmune disease in a subject by administering to the subject an effective amount of a therapeutic agent that increases the population of protective CD4g13 T cells, methods for detecting a protective or pathological immune response and methods for stimulating a protective CD4g13 T cell-mediated immune response to a cell population or a local tissue or organ in a subject in need thereof. The invention further includes a kit for diagnosing a pathological or protective g13Th1 or g13Th2 T cell responses in a subject.

### CROSS-REFERENCE TO RELATED APPLICATIONS

The present application claims priority under 35 U.S.C. § 119(e) to U.S. Provisional Patent Application 62/811,149, filed Feb. 27, 2019, which application is incorporated herein by reference in its entirety.

### STATEMENT REGARDING FEDERALLY SPONSORED RESEARCH OR DEVELOPMENT

This invention was made with government support under grant AI113103, awarded by the National Institutes of Health. The government has certain rights in the invention.

### BACKGROUND OF THE INVENTION

Surveillance and defense of the enormous mucosal interface with the nonsterile world is critical to protecting the host from a wide range of pathogens. Chlamydia trachomatis (Ct) is an intracellular bacterial pathogen that replicates almost exclusively in the epithelium of the reproductive tract. The first line of defense from chlamydial genital infection is the mucosal barrier of the reproductive tract. The first and most important immune response to Chlamydia infection is a local one, whereby immune cells such as leukocytes are recruited to the site of infections, and subsequently secrete pro-inflammatory cytokines and chemokines. Immune cells also work to initiate and potentiate chronic inflammation through the production of reactive oxygen species, and the release of molecules with degradative properties (e.g. defensins, cathepsins, and lysozyme). Long-term inflammation can lead to cell proliferation, tissue remodeling, and scarring, as well as onset of autoimmune responses in genetically disposed individuals.

Ct infections of the reproductive tract have evaded public health interventions for the past several decades. In the United States and Canada, the incidence of Ct infections continues to climb despite effective antibiotics, and public health measures that increased screening, partner notification, and treatment. In fact, the attempt to control Ct infection likely aborts the development of herd immunity and results in the need to treat even great numbers of individuals; arrested immunity due to doxycycline treatment is demonstrable in the C. muridarum mouse model. It is widely accepted by researchers and public health officials that the only intervention likely to reduce the incidence of disease and the human toll and expense inflicted by Ct-induced infertility and ectopic pregnancy is a Chlamydia vaccine. While much progress has been made, the immunologic goals of a Chlamydia vaccine remain elusive and no human vaccine against the urogenital serovars has been attempted. The finding that untreated humans can self-clear genital tract infections, and that those who do are less likely to be re-infected provides proof-in-principal for a Chlamydia genital tract vaccine.

The immunologic goal of vaccination for protective immunity against urogenital serovars is likely a multifunctional Th1 response. The role of antibodies in a future Ct vaccine is unclear, with animal model data supporting and refuting a role for Chlamydia-specific antibodies in protective immunity absent a pre-existing T cell response. In human studies in IgG and IgA antibody responses measured in serum do not correlate with protective immunity, and a prospective human clinical investigation showed a linear positive correlation between anti-chlamydial antibody titers and future infertility. In mice CD8 T cell responses are associated with immunopathology rather than protection; though there are caveats to this statement including evidence for CD8 protection with a trachoma vaccine in macaques, and the identification of CD8 epitopes that correlate with self-resolution in humans. While many questions remain about the pathophysiology of protection versus immunopathology it is generally accepted that the reliably protective arm of the adaptive immune response is the CD4 T cell response. A critical component for rational vaccine development is a surrogate biomarker for protective immunity.

A practicable surrogate biomarker for protective immunity is defined as a testable parameter that can be reasonably and reliably measured after administration of a vaccine that correlates with resistance to infection. Currently there are only two such surrogate biomarkers for Ct immunity: (1) A PBMC IFN-γ (also referred to as “IFN-g”) response to Chlamydia HSP60 that is not useful in the context of vaccines as HSP60 is an unlikely candidate component of a subunit vaccine, and (2) a PBMC IL-13 response to EB (elementary body; infectious form of Ct). The latter has been an enigma as IL-13 is a Th2 cytokine, and Th2 responses are associated with negative outcomes in animal models of Chlamydia infection.

Clearly, there is an urgent need in the art for identifying the key components and biomarkers of mucosal immunity. Furthermore there is a need in the art for compositions and methods for treating and preventing Chlamydia's infections and more generally for treating inflammatory and autoimmune diseases connected to the mucosal immune system. This invention addresses this need.

### SUMMARY OF THE INVENTION

In one aspect, the invention provides a method for treating an inflammatory or autoimmune disease in a subject, the method comprising administering to the subject an effective amount of a therapeutic agent that increases the population of protective g13Th1 T cells in the subject, thereby treating inflammatory or autoimmune disease in the subject.

In another aspect, the invention provides a method for treating an inflammatory or autoimmune disease in a subject, the method comprising administering to the subject an effective amount of a therapeutic agent that decreases the population of pathologic g13Th1 T cells in the subject, thereby treating inflammatory or autoimmune disease in the subject.

In yet another aspect, the invention provides a method for treating an inflammatory or autoimmune disease in a subject, the method comprising administering to the subject an effective amount of a therapeutic agent that increases the population of protective g13Th2 T cells in the subject, thereby treating inflammatory or autoimmune disease in the subject.

In yet another aspect, the invention provides a method for treating an inflammatory or autoimmune disease in a subject, the method comprising administering to the subject an effective amount of a therapeutic agent that decreases the population of pathologic g13Th2 T cells in the subject, thereby treating inflammatory or autoimmune disease in the subject.

In yet another aspect, the invention provides a method of assessing the type of immune response in an inflammatory or autoimmune disease in a subject, the method comprising:
detecting the level of cytokines IL2, IL13, IL4, IL5, IL17 and IL22 in a sample from the subject;
determining the level of gene expression of at least one T cell biomarker selected from the group consisting of, Cd93, Large, Cpa3, Pde8a, Pgr, Nm1, Dapk1, Cyp4f39, Noxred1, Treml2, Fam213a, Bmp8, Lrrc32, Cyp11a1, tarm1, Chat2, Chil3, Gpm6b, Bace2, Lag3, Acbd7, Ctse, Hsd17b11 and Hrh4 in a sample from the subject;
comparing the level of the cytokines or the at least one T cell biomarker in the sample from the subject to a baseline level in a control subject not having an inflammatory or autoimmune disease, wherein a higher level of the cytokines or the at least one T cell biomarker in the sample as compared to the level of the cytokines or the at least one T cell biomarker in the control is indicative of a g13Th1 or g13Th2 T cell response; and,
wherein when a g13Th1 or g13Th2 T cell response is indicated, treatment of the inflammatory or autoimmune disease is recommended.

In yet another aspect, the invention provides a method of detecting a pathological T cell response in an inflammatory or autoimmune disease in a subject, the method comprising: determining the presence of a pathological g13Th1 T cell by detecting the level of cytokines IL2, IL13, IL17, IL22 and IFNg in a sample from the subject;
determining the level of gene expression of at least one pathological g13Th1 T cell biomarker selected from the group consisting of: Cd93, Large, Cpa3, Pde8a, Pgr, Nm1, Dapk1, Cyp4f39, Noxred1, Treml2;
comparing the level of the cytokines or the at least one g13Th1 T cell biomarker in the sample from the subject to a baseline level in a control subject not having an inflammatory or autoimmune disease, wherein a higher level of the cytokines or the at least one g13Th1 T cell biomarker in the sample as compared to the level of the cytokines or the at least one g13Th1 T cell biomarker in the control is indicative of a pathological T cell response; and,
wherein when a pathological T cell response is indicated, treatment of the inflammatory or autoimmune disease is recommended.

In yet another aspect, the invention provides a method of detecting a pathological T cell response in an inflammatory or autoimmune disease in a subject, the method comprising: determining the presence of a pathological g13Th2 T cell by detecting the level of cytokines IL2, IL13, IL4, IL5 and IFNg in a sample from the subject;
determining the level of gene expression of at least one g13Th2 T cell biomarker selected from the group consisting of: Fam213a, Bmp8, Lrrc32, Cyp11a1, tarm1, Chat2, Chil3, Gpm6b, Bace2, Lag3, Acbd7, Ctse, Hsd17b11 and Hrh4;
comparing the level of the cytokines or the at least one g13Th2 T cell biomarker in the sample from the subject to a baseline level in a control subject not having an inflammatory or autoimmune disease, wherein a higher level of the cytokines or the at least one g13Th2 T cell biomarker in the sample as compared to the level of the cytokines or the at least one g13Th2 T cell biomarker in the control is indicative of a pathological T cell response; and,
wherein when a pathological T cell response is indicated, treatment of the inflammatory or autoimmune disease is recommended.

In yet another aspect, the invention provides a method of detecting a protective T cell response in an inflammatory or autoimmune disease in a subject, the method comprising: determining the presence of a protective g13Th1 T cell by detecting the level of cytokines IL2, IL13, IL17, IL22 and IFNg in a sample from the subject;
determining the level of gene expression of at least one g13Th1 T cell biomarker selected from the group consisting of: Cd93, Large, Cpa3, Pde8a, Pgr, Nm1, Dapk1, Cyp4f39, Noxred1 and Treml2;

comparing the level of the cytokines or the at least one g13Th1 T cell biomarker in the sample from the subject to a baseline level in a control subject not having an inflammatory or autoimmune disease, wherein a higher level of the cytokines or the at least one g13Th1 T cell biomarker in the sample as compared to the level of the cytokines and/or to the at least one g13Th1 T cell biomarker in the control is indicative of a protective T cell response; and, wherein when a protective T cell response is indicated, treatment of the inflammatory or autoimmune disease is recommended.

In yet another aspect, the invention provides a method of detecting a protective T cell response in an inflammatory or autoimmune disease in a subject, the method comprising:
determining the presence of a protective g13Th2 T cell by detecting the level of cytokines IL2, IL13, IL4, IL5 and IFNg in a sample from the subject;
determining the level of gene expression of at least one g13Th2 T cell biomarker selected from the group consisting of: Fam213a, Bmp8, Lrrc32, Cyp11a1, tarm1, Chat2, Chil3, Gpm6b, Bace2, Lag3, Acbd7, Ctse, Hsd17b11 and Hrh4;
comparing the level of the cytokines or the at least one g13Th2 T cell biomarker in the sample from the subject to a baseline level in a control subject not having an inflammatory or autoimmune disease, wherein a higher level of the cytokines or the at least one g13Th2 T cell biomarker in the sample as compared to the level of the cytokines and/or to the at least one g13Th2 T cell biomarker in the control is indicative of a protective T cell response; and, wherein when a protective T cell response is indicated, treatment of the inflammatory or autoimmune disease is recommended.

In yet another aspect, the invention provides a method for stimulating a protective CD4g13 T cell-mediated immune response to a cell population or a local tissue or organ in a subject in need thereof, the method comprising administering to the subject an effective amount of a therapeutic agent that increases the population of protective CD4g13 T cells and a pharmaceutical acceptable carrier.

In yet another aspect, the invention provides a kit for diagnosing a g13Th1 T cell response in an inflammatory or autoimmune disease in a subject, the kit comprising a plurality of oligonucleotides that are configured to detect at least one biomarker selected from the group consisting of Cd93, Large, Cpa3, Pde8a, Pgr, Nm1, Dapk1, Cyp4f39, Noxred1 and Treml2.

In yet another aspect, the invention provides a kit for diagnosing a g13Th2 T cell response in an inflammatory or autoimmune disease in a subject, the kit comprising a plurality of oligonucleotides that are configured to detect at least one biomarker selected from the group consisting of: Fam213a, Bmp8, Lrrc32, Cyp11a1, tarm1, Chat2, Chil3, Gpm6b, Bace2, Lag3, Acbd7, Ctse, Hsd17b11 and Hrh4.

In yet another aspect, the invention provides a pharmaceutical composition for treating an inflammatory or autoimmune disease in a subject, the pharmaceutical composition comprising a therapeutic agent that increases the population of protective g13Th1 T cells and a pharmaceutical acceptable carrier.

In yet another aspect, the invention provides a pharmaceutical composition for treating an inflammatory or autoimmune disease in a subject, the pharmaceutical composition comprising a therapeutic agent that increases the population of protective g13Th2 T cells and a pharmaceutical acceptable carrier.

In yet another aspect, the invention provides a pharmaceutical composition for treating an inflammatory or autoimmune disease in a subject, the pharmaceutical composition comprising a therapeutic agent that decreases the population of pathological g13Th1 cells and a pharmaceutical acceptable carrier.

In yet another aspect, the invention provides a pharmaceutical composition for treating an inflammatory or autoimmune disease in a subject, the pharmaceutical composition comprising a therapeutic agent that decreases the population of pathological g13Th2 cells and a pharmaceutical acceptable carrier.

In yet another aspect, the invention provides a method of assessing a response to a therapeutic agent in a subject, the method comprising:
administering an effective amount of the therapeutic agent to the subject;
determining the presence of a protective g13Th1 T cell by detecting the level of cytokines IL2, IL13, IL17, IL22 and IFNg in a sample from the subject;
determining the level of gene expression of at least one g13Th1 T cell biomarker selected from the group consisting of: Cd93, Large, Cpa3, Pde8a, Pgr, Nm1, Dapk1, Cyp4f39, Noxred1 and Treml2;
comparing the level of the cytokines or the at least one g13Th1 T cell biomarker in the sample from the subject to a baseline level in a control subject not having an inflammatory or autoimmune disease,
wherein a higher level of the cytokines or the at least one protective g13Th1 T cell biomarker in the sample as compared to the level of the cytokines and/or to the at least one g13Th1 T cell biomarker in the control is indicative of a protective, therapeutic agent-generated, T cell response.

In yet another aspect, the invention provides a method of assessing a response to a therapeutic agent in a subject, the method comprising:
administering an effective amount of therapeutic agent to the subject;
determining the presence of a protective g13Th2 T cell by detecting the level of cytokines IL2, IL13, IL4, IL5 and IFNg in a sample from the subject;
determining the level of gene expression of at least one g13Th2 T cell biomarker selected from the group consisting of: Fam213a, Bmp8, Lrrc32, Cyp11a1, tarm1, Chat2, Chil3, Gpm6b, Bace2, Lag3, Acbd7, Ctse, Hsd17b11 and Hrh4;
comparing the level of the cytokines or the at least one g13Th2 T cell biomarker in the sample from the subject to a baseline level in a control subject not having an inflammatory or autoimmune disease, wherein a higher level of the cytokines or the at least one g13Th2 T cell biomarker in the sample as compared to the level of the cytokines and/or to the at least one g13Th2 T cell biomarker in the control is indicative of a protective, therapeutic agent-generated, T cell response.

In certain embodiments, the therapeutic agent increases the population of protective g13Th1 T cells, an increase in the level of at least one cytokine selected from the group consisting of IL17 and IL22 is detected.

In certain embodiments, the population of protective g13Th1 T cells comprise at least one biomarker selected from the group consisting of: Cd93, Large, Cpa3, Pde8a, Pgr, Nm1, Dapk1, Cyp4f39, Noxred1 and Treml2.

In certain embodiments, the therapeutic agent decreases the population of pathological g13Th2 T cells thereby decreasing the level of at least one cytokine selected from the group consisting of IL4 and IL5.

In certain embodiments, the population of pathological g13Th2 T cells comprises at least one biomarker selected from the group consisting of: Fam213a, Bmp8, Lrrc32, Cyp11a1, tarm1, Chat2, Chil3, Gpm6b, Bace2, Lag3, Acbd7, Ctse, Hsd17b11 and Hrh4.

In certain embodiments, the therapeutic agent decreases the population of pathologic g13Th1 T cells, a decrease in the level of at least one cytokine selected from the group consisting of IL17 and IL22 is detected.

In certain embodiments, the population of pathologic g13Th1 T cells comprise at least one biomarker selected from the group consisting of: Cd93, Large, Cpa3, Pde8a, Pgr, Nm1, Dapk1, Cyp4f39, Noxred1 and Treml2.

In certain embodiments, the therapeutic agent increases the population of protective g13Th2 T cells thereby increasing the level of at least one cytokine selected from the group consisting of IL4 and IL5.

In certain embodiments, the population of protective g13Th2 T cells comprises at least one biomarker selected from the group consisting of: Fam213a, Bmp8, Lrrc32, Cyp11a1, tarm1, Chat2, Chil3, Gpm6b, Bace2, Lag3, Acbd7, Ctse, Hsd17b11 and Hrh4.

In certain embodiments, the therapeutic agent is at least one selected from the group consisting of a large molecule, a small molecule, a ligand, an enzyme, a peptidomimetic, an antibody, an aptamer, a vaccine and a combination thereof.

In certain embodiments, the sample is selected from the group consisting of blood, endometrial biopsy, stool and synovial fluid.

In certain embodiments, the population of protective CD4g13 T cells consists of at least one selected from the group consisting of protective g13Th1 cells and protective g13Th2 cells.

In certain embodiments, the inflammatory or autoimmune disease is linked to at least one infection selected from the group consisting of bacterial, viral and parasitic.

In certain embodiments, the bacterial infection is a Chlamydia bacterium.

In certain embodiments, the inflammatory or autoimmune disease is selected from the group consisting of mucosal inflammation, orchitis, epididymis, inflammatory bowel disease, Crohn's disease, ulcerative colitis, multiple sclerosis, rheumatoid arthritis, psoriasis.

In certain embodiments, the therapeutic agent is at least one selected from the group consisting of a large molecule, a small molecule, a ligand, an enzyme, a peptidomimetic, an antibody, an aptamer, a vaccine, and a combination thereof.

In certain embodiments, the subject is a mammal. In certain embodiments, the mammal is a human.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1B are series of flow cytometry plots depicting B cell dynamics and antigen presentation in the genital tract during C. muridarum infections. FIG. 1A: Single cells suspensions of genital tracts from the following conditions were gated on CD79a (B cells) and analyzed for level of B220; B220 high=B lymphocytes; B220 low=plasma B cells. Uninfected mice (Naive), day 7 primary infection (D7_pri_inf), day 35 primary infection (D35_pri_Cm inf), and day 5 secondary infection (D5_sec_inf) were investigated. Mice were pre-treated with medroxyprogesterone and infected 1 week later with 1500 IFU of C. muridarum; representative data shown from a minimum of 2 experiments for each experimental group. FIG. 1B: Analysis of B cell-APC-derived and immune splenocyte-derived Chlamydia-specific polyclonal T cell lines for production of IL-13. T cell lines were activated for 5 h with PMA/ionomycin/brefeldin A/monensin and stained for CD8 vs IL-13. CD4 T cells are identified as CD8neg in this assay (see materials and methods).

FIGS. 2A-2D are series of graphs illustrating the cytokine profile of B-cell derived polyclonal T cell line B2 versus immune splenocyte-derived polyclonal CD4 T cell lines and the presence of CD4g13 T cells in the genital tract and spleen. FIG. 2A: Polyclonal B-cell derived line #2(B2) and splenocyte-derived line #2 (sp12) were activated for 5 h with PMA/ionomycin/brefeldin A/monensin then stained for CD8 (negative stain for CD4 T cells) vs IL-13, and IFN-g vs IL-13. FIG. 2B: Specificity and cytokine profiles for B2 and five splenocyte-derived T cell lines (sp11-5); 2.5×10⁴ T cells were co-incubated with 5×10⁴ purified immune B cells unpulsed (APC) or pulsed with uvMoPn (Ag) in triplicate. Cell culture supernatants harvested at 48 h and IFN-g, IL-13, and IL-4 determined by ELISA. Comparisons between control (Con) and antigen-pulsed (Ag) B cell activation for each cytokine and T cell line; *=p value<0.05,****=p value<0.0005. FIG. 2C: Frequency of IL-13+CD4 T cells in the spleen and genital tract. C57BL/6 female mice in experimental groups of 4 mice each were PmpG immunized (PmpG Imm), unimmunized (Prim. Infect), or previously infected with C. muridarum (1500 IFU/vaginally; Sec. Infect). 4 weeks after last immunization or C. muridarum primary infection mice were challenged with intravaginally with C. muridarum. Six days later single cell suspensions prepared from individual mice (except naive uninfected control) and CD4 T cells were scored for IFN-γ (also referred to as “IFN-g”), TNFα (also referred to as “TNFa”), IL-13, and IFN-γ/IL-13. FIG. 2D: For genital tract CD4g13 T cells the first panel shows the % that also produce IFN-γ (left panel) and their intensity of IFN-γ production (right panel). Data for individual mice shown in FIG. 11 and FIG. 12.

FIGS. 3A-3C are series graphs illustrating cytokine profiles of Chlamydia-specific CD4 clones, IL-13 production, and replication control. FIG. 3A: 2.5×10⁴ T cells were activated with immobilized anti-CD3 antibody in 96-well plates; 5×10⁴ T cells/well were used for IL-4 to increase sensitivity. 20 h culture supernatants were analyzed for IL-2 (yellow), IFN-g (black), IL-13 (green), IL-10 (gray hatched), TNFa (dark blue), IL-17 (red), IL-22 (light blue), IL-5 (orange), and IL-4 (pink). All visible bars are significant (p values<0.05) compared to parallel wells lacking anti-CD3 antibodies. Data presented are aggregated from two independent experiments. FIG. 3B: CD4g13 IL-13 pathway is partially calcineurin-independent. 2.5×10⁴ T cells were activated by immobilized anti-CD3 in the absence and presence of 500 nM (2 ng/ml) CsA without or with small molecule inhibitors of CrTh2 (CrTh2-1, CrTh2-2) at 5 nM (˜50×IC₅₀). Top panel IL-13 production, middle panel IFN-g production, bottom panel relative IL-13 vs IFN-g in the presence of 500 nM CsA. Aggregated data from two independent experiments; **=p value<0.005; ***=p value<0.0005; ****=p value<0.00005. FIG. 3C: Termination of C. muridarum replication. C57epi.1 epithelial cells untreated (top) or pretreated with 10 ng/ml IFN-g for 6 h (middle), were washed then infected with 2 IFU C. muridarum per cell. 4 h later 1.5×10⁵ T cells (1:1 ratio) were added to each well and to uninfected wells as controls. 28 h later 100 ul supernatant was removed for IFN-g analysis and wells harvested into SPG buffer. IFN-g levels determined by ELISA; IFU number by culture on McCoy cells. All IFN-g (pg/ml) in parentheses are significant (p value<0.05); IFN-g levels for all CD4 clones cultured on uninfected epithelial cells were <100 pg/ml. Aggregated data from two independent experiments. Cytolytic capability of the CD4 clones versus a Chlamydia-specific CD8 T cell clone in redirected lysis (bottom panel). 10,000 T cells were incubated with 10,000 Fc receptor-bearing P815 cells in the absence (spontaneous release) and presence of 0.5 ug/ml anti-CD3 antibody (activation/lysis) in quadruplicate in a 4 h assay based on LDH release. % specific lysis is shown for each of the T cell clones; single experiment done as quadruplicates.

FIG. 4 is a heat map depicting the clustering of the top 1000 genes by the ANOVA p-value, i.e. genes that were differentially expressed in at least one clone. This clustering shows the two CD4g13 T cell clones sBT13-7 and sBT16-8 (top two vertical bars on left) are the most alike among the six CD4 T cell clones in the panel (left to right: blue-red-blue-red-blue pattern).

FIG. 5 is a series of immunoblots showing the protein levels of selected differentiation/transcription factors. On day 5 of the usual culture cycle the indicated CD4 T cell clones were purified by ficoll-hypaque, whole cell lysates prepared and utilized for immunoblotting for Tbet, Gata3, Eomes, and Fhl2. Blots were stripped and re-probed with anti-βactin as the loading control. Molecular weight markers in kilo Daltons are indicated on the right margin.

FIGS. 6A-6D are series of graphs and images depicting adoptive transfer: Bacterial shedding and pathology scoring. FIG. 6A: IFU shedding in the lower genital tract. FIG. 6B: % of mice shedding at each time point. FIG. 6C: On day 56 mice were killed and uterine and oviduct pathology was scored in situ (see materials and methods). FIG. 6D: Digital images of the oviducts; black arrows indicate scored pathology. *=p value<0.05; **=p value<0.005; ***=p value<0.0005. Aggregated data from 3 experiments; control (14 mice), 4uvmo-3 (9 mice), sBT13-7 (9 mice), sBT16-8 (8 mice). Txf=adoptive transfer.

FIG. 7 is a series of flow cytometry plots showing the gating strategy of CD79 staining to identify B cells in mouse genital tracts. Single cell suspensions of genital tracts were surface stained for B220 (FITC) with the viability dye (aqua fluorescent reactive dye), followed by intracellular staining for CD79a (eFluor660). The CD79 (−) control (CD79 minus staining) was included.

FIG. 8 is a series of flow cytometry plots showing CD4 PE vs CD8 FITC staining of polyclonal T cell lines derived using immune-B-cell-APC and unfractionated immune-splenoctye-APC after three passages.

FIG. 9 is a histogram depicting polyclonal T cell lines with fading IL-13 production cultured as per usual protocol and, in parallel, the usual protocol plus 10 ng/ml TGFβ1. At the end of one week the T cell lines were activated with immobilized anti-CD3 and IL-13 levels measured in culture supernatant. With the exception of B2 all the lines made more IL-13 after a week in TGFβ1. It was also noted that expansions in TGFβ1 conditions yielded˜50% more cells per well.

FIG. 10 is a series of flow cytometry plots showing the spots and gating strategy to identify Chlamydia-specific IFN-γ/IL-13 producing CD4 T cells (CD4γ13 T cells) in genital tract from a representative mouse after secondary Cm infection. Single cell suspensions of genital tracts were stimulated with dead EB and surface stained for CD3, CD4, CD8 with the viability dye, followed by cytokine intracellular staining.

FIG. 11 is a table listing the frequency of CD4IL-13 T cells in the spleen. The Intracellular cytokine profiles is shown for splenic CD4 T cells in naïve cells (these were pooled to generate single data point). This profiling was done in PmpG vaccination following a primary infection, in a primary infection of naïve mice, and in secondary infection. Single cell suspensions were prepared from individual mice (except naïve uninfected control) on day 6 post infection and CD4 T cells scored for IFN-γ, TNFα, IL-13, and IFN-γ/IL-13.

FIG. 12 is a table listing the frequency of CD4IL-13 T cells in the genital tract (GT). The Intracellular cytokine profiles is shown for genital tract CD4 T cells in naïve cells (these were pooled to generate single data point). This profiling was done in PmpG vaccination following a primary infection, in a primary infection of naïve mice, and in secondary infection. Single cell suspensions were prepared from individual mice (except naïve uninfected control) on day 6 post infection and CD4 T cells scored for IFN-γ, TNFα, IL-13, and IFN-γ/IL-13.

FIG. 13 is a drawing of the systemic and mucosal immunity pathway. In the mucosal pathway, during Chlamydia infections, gTh2 cells represent pathological T cell subset and gTh1 cells represent protective T cell subset which could be relevant for vaccine development.

FIG. 14 shows representative flow plots of PBMCs from a healthy human donor. Resting PBMCs were stained with antibodies against CD4, CD93 (to identify CD4g13 Th1 cells), and GPm6B (to identify CD4g13 Th2 cells).

FIG. 15 illustrates that CD93 and Gpm6b are mutually exclusive. FIG. 15 shows plots for staining of endometrial mononuclear cells gated on CD3 and CD4 and for analyzing expression of CD93 (g13Th1) versus Gpm6b (g13Th2). In this one individual, recruited without knowledge of any specific pathological condition, the dominant CD4g13 subset was Gpm6b positive (g13Th2), representing 9.26% of endometrial CD4 T cells. There were no detectable CD93 positive CD4 T cells. There are no double positive CD93/Gpm6b cells, supporting that these are subset specific biomarkers.

### DETAILED DESCRIPTION

The invention includes compositions for manipulating the CD4 tissue resident memory T cells (TRM) immune compartment. The compositions comprise a therapeutic agent that decreases the population of pathological CD4g13 T cells (also referred to as “CD4γ13”) and a therapeutic agent that increases the population of protective CD4g13 T cells. The invention also includes methods for treating an inflammatory or autoimmune disease in a subject by administering to the subject an effective amount of a therapeutic agent that increases the population of protective CD4g13 T cells in the subject, methods for detecting a protective or pathological immune response and methods for stimulating a protective CD4g13 T cell-mediated immune response to a cell population or a local tissue or organ in a subject in need thereof. The invention further includes a kit for diagnosing a pathological or protective CD4g13 T cell response in a subject.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice of and/or for the testing of the present invention, the preferred materials and methods are described herein. In describing and claiming the present invention, the following terminology will be used according to how it is defined, where a definition is provided.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, in some instances ±5%, in some instances ±1%, and in some instance ±0.1% from the specified value, as such variations are appropriate to perform the disclosed methods.

The term “antibody,” as used herein, refers to an immunoglobulin molecule binds with an antigen. Antibodies can be intact immunoglobulins derived from natural sources or from recombinant sources and can be immunoreactive portions of intact immunoglobulins. Antibodies are typically tetramers of immunoglobulin molecules. The antibody in the present invention may exist in a variety of forms where the antibody is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

The term “high affinity” as used herein refers to high specificity in binding or interacting or attraction of one molecule to a target molecule.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, “aptamer” refers to a small molecule that can bind specifically to another molecule. Aptamers are typically either polynucleotide- or peptide-based molecules. A polynucleotide aptamer is a DNA or RNA molecule, usually comprising several strands of nucleic acids, that adopts highly specific three-dimensional conformation designed to have appropriate binding affinities and specificities towards specific target molecules, such as peptides, proteins, drugs, vitamins, among other organic and inorganic molecules. Such polynucleotide aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

As used herein, “sample” or “biological sample” refers to anything, which may contain an analyte (e.g., polypeptide, polynucleotide, or fragment thereof) for which an analyte assay is desired. The sample may be a biological sample, such as a biological fluid or a biological tissue. In one embodiment, a biological sample is a endometrial sample. Such a sample may include diverse cells, proteins, and genetic material. Examples of biological tissues also include organs, tumors, lymph nodes, arteries and individual cell(s). Examples of biological fluids include urine, blood, plasma, serum, saliva, semen, stool, sputum, cerebral spinal fluid, synovial fluid, tears, mucus, amniotic fluid or the like.

The term “biopsy” refers to a specimen obtained by removing tissue from living patients for diagnostic examination. The term includes aspiration biopsies, brush biopsies, chorionic villus biopsies, endoscopic biopsies, excision biopsies, needle biopsies (specimens obtained by removal by aspiration through an appropriate needle or trocar that pierces the skin, or the external surface of an organ, and into the underlying tissue to be examined), open biopsies, punch biopsies (trephine), shave biopsies, sponge biopsies, and wedge biopsies. Biopsies also include a fine needle aspiration biopsy, a minicore needle biopsy, and/or a conventional percutaneous core needle biopsy.

As used herein, the term “biomarker” includes a polynucleotide or polypeptide molecule which is present or increased in quantity or activity in a subject having an inflammatory or auto-immune disease.

The term “limited toxicity” as used herein, refers to the peptides, polynucleotides, cells and/or antibodies of the invention manifesting a lack of substantially negative biological effects, anti-tumor effects, or substantially negative physiological symptoms toward a healthy cell, non-tumor cell, non-diseased cell, non-target cell or population of such cells either in vitro or in vivo.

The term “autoimmune disease” as used herein is defined as a disorder or condition that results from an antibody mediated autoimmune response against autoantigens. An autoimmune disease results in the production of autoantibodies that are inappropriately produced and/or excessively produced to a self-antigen or autoantigen.

As used herein, the term “autologous” is meant to refer to any material derived from the same individual to which it is later to be re-introduced into the individual.

“Co-stimulatory ligand,” as the term is used herein, includes a molecule on an antigen presenting cell (e.g., an APC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

“Effective amount” or “therapeutically effective amount” are used interchangeably herein, and refer to an amount of a compound, formulation, material, or composition, as described herein effective to achieve a particular biological result. Such results may include, but are not limited to, the inhibition of virus infection as determined by any means suitable in the art.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by a promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes), retrotransposons (e.g. piggyback, sleeping beauty), and viruses (e.g., lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an Arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

“Intracellular domain” refers to a portion or region of a molecule that resides inside a cell.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “proinflammatory cytokine” refers to a cytokine or factor that promotes inflammation or inflammatory responses. Examples of proinflammatory cytokines include, but are not limited to, chemokines (CCL, CXCL, CX3CL, XCL), interleukins (such as, IL-1, IL-2, IL-3, IL-5, IL-6, IL-7, IL-9, IL-10 and IL-15), interferons (IFNγ), and tumor necrosis factors (TNFα and TNFβ).

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

“Signaling domain” refers to the portion or region of a molecule that recruits and interacts with specific proteins in response to an activating signal.

The term “subject” is intended to include living organisms in which an immune response can be elicited (e.g., mammals).

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cells that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

“Transmembrane domain” refers to a portion or a region of a molecule that spans a lipid bilayer membrane.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses.

Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

By the term “specifically binds,” as used herein, is meant an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates to the discovery of a subset of CD4 T cells that belong to the mucosal TRM T cells relevant to chronic inflammatory events in mucosa, and likely dermis and brain, that have an IL-13 signature. These cells produce specific cytokines IFN-g, IL-2 and IL-13 and are referred to herein as CD4g13 T cells (or also as “CD4γ13”). Detailed analysis of these subset of T cells classifies them in two subgroups based on secondary cytokines and associated biomarkers. CD4g13 T cells are further defined by production of IL-17/IL-22 (g13Th1) or IL-4/IL-5 (g13Th2). In specific disease states CD4g13 T cell subsets can be either protective or pathologic. During Chlamydia infections, g13Th1 are protective, while g13Th2 are non-protective and likely pathologic. In multiple sclerosis, IL-13 levels in the CSF are neuroprotective; in psoriasis, the IL-13/IL-4 locus is associated with pathology. Chronic inflammatory bowel disease has a g13Th2 profile at the tissue level implying a role in pathologic inflammation. Knowledge of unique biomarkers for g13Th1 and g13Th2 cells permits independent therapeutic manipulation of these cells as their roles in specific inflammatory diseases is defined over time.

### Methods of the Invention

In one aspect, the present invention is directed to a method of treating an inflammatory or autoimmune disease in a subject. The method comprises administering to the subject an effective amount of a therapeutic agent that increases or decreases the population of CD4g13 T cells of the g13Th1 or g13Th2 phenotype, thereby enhancing immunity against intracellular microbial pathogens in the context of a vaccine, or treating infection associated pathologic inflammation or autoimmune disease in the subject.

In one aspect, the subject is a mammal, and preferably a human. Non-limiting examples of inflammatory and autoimmune diseases contemplated by the invention are diseases associated with mucosal immunity. In one embodiment, the inflammatory and autoimmune disease is associated with at least one infection selected from the group consisting of bacterial, viral and parasitic. In some embodiments, the bacterial infection is a Chlamydia bacterium. In another embodiment, the inflammatory and autoimmune disease is selected from the group consisting of mucosal inflammation, orchitis, epididymis, inflammatory bowel disease, Crohn's disease, ulcerative colitis, multiple sclerosis, rheumatoid arthritis and psoriasis.

### Therapeutic Agent

The invention contemplates use of a therapeutic agent that either increases the population of protective CD4g13 T cells (i.e. protective g13Th1 or g13Th2 T cells) or decreases the population of pathological CD4g13 T cells (i.e. pathological g13Th1 or g13Th2 T cells).

In one embodiment, when the therapeutic agent increases the population of protective g13Th1 T cells, an increase in the level of at least one cytokine selected from the group consisting of IL17 and IL22 is detected. In another embodiment, the population of g13Th1 T cells comprise at least one biomarker selected from the group consisting of: Cd93, Large, Cpa3, Pde8a, Pgr, Nm1, Dapk1, Cyp4f39, Noxred1 and Treml2.

In another embodiment, when the therapeutic agent decreases the population of pathologic g13Th1 T cells, a decrease in the level of at least one cytokine selected from the group consisting of IL17 and IL22 is detected. In another embodiment, the population of g13Th1 T cells comprise at least one biomarker selected from the group consisting of: Cd93, Large, Cpa3, Pde8a, Pgr, Nm1, Dapk1, Cyp4f39, Noxred1 and Treml2.

In another embodiment, when the therapeutic agent increases the population of protective g13Th2 T cells, an increase in the level of at least one cytokine selected from the group consisting of IL4 and IL5 is detected. In yet another embodiment, the population of protective g13Th2 T cells comprises at least one biomarker selected from the group consisting of: Fam213a, Bmp8, Lrrc32, Cyp11a1, tarm1, Chat2, Chil3, Gpm6b, Bace2, Lag3, Acbd7, Ctse, Hsd17b11 and Hrh4.

In another embodiment, the therapeutic agent decreases the population of pathological g13Th2 T cells thereby decreasing the level of at least one cytokine selected from the group consisting of IL4 and IL5. In yet another embodiment, the population of pathological g13Th2 T cells comprises at least one biomarker selected from the group consisting of: Fam213a, Bmp8, Lrrc32, Cyp11a1, tarm1, Chat2, Chil3, Gpm6b, Bace2, Lag3, Acbd7, Ctse, Hsd17b11 and Hrh4.

In one embodiment, the CD4g13 T cell's therapeutic agent is at least one selected from the group consisting of a large molecule, a small molecule, a ligand, an enzyme, a peptidomimetic, an antibody, an aptamer, a vaccine and a combination thereof.

In some embodiments, the therapeutic agent is any small or large molecule activator or inhibitor known in art to interact fully or partially with CD4g13 T cells' activity.

In certain embodiments an aptamer is used. Aptamers are macromolecules composed of nucleic acid that bind tightly to a specific molecular target. Tuerk and Gold (Science, 1990, 249:505-510) discloses SELEX (Systematic Evolution of Ligands by Exponential Enrichment) method for selection of aptamers. In the SELEX method, a large library of nucleic acid molecules (e.g., 1015 different molecules) is produced and/or screened with the target molecule. Isolated aptamers can then be further refined to eliminate any nucleotides that do not contribute to target binding and/or aptamer structure (i.e., aptamers truncated to their core binding domain). See, e.g., Jayasena, 1999, Clin. Chem. 45:1628-1650 for review of aptamer technology.

In other embodiments, the therapeutic agent is an antibody. In certain embodiments, the antibody comprises an antibody selected from a polyclonal antibody, a monoclonal antibody, a humanized antibody, a synthetic antibody, a heavy chain antibody, a human antibody, and a biologically active fragment of an antibody and any combination thereof. Methods of producing antibodies are known in the art. It will be appreciated by one skilled in the art that an antibody comprises any immunoglobulin molecule, whether derived from natural sources or from recombinant sources, which is able to specifically bind to an epitope present on a target molecule. Antibodies may be generated in this manner in several non-human mammals such as, but not limited to goat, sheep, horse, camel, rabbit, and donkey. Methods for generating polyclonal antibodies are well known in the art and are described, for example in Harlow et al., 1998, In: Antibodies, A Laboratory Manual, Cold Spring Harbor, NY.

The present invention also can include the use of humanized antibodies specifically reactive with an epitope present on a target molecule. These antibodies are capable of binding to the target molecule. The humanized antibodies useful in the invention have a human framework and have one or more complementarity determining regions (CDRs) from an antibody, typically a mouse antibody, specifically reactive with a targeted cell surface molecule. When the antibody used in the invention is humanized, the antibody can be generated as described in Queen et al. (U.S. Pat. No. 6,180,370), Wright et al., 1992, Critical Rev. Immunol. 12(3,4):125-168, and in the references cited therein, or in Gu et al., 1997, Thrombosis & Hematocyst 77(4):755-759, or using other methods of generating a humanized antibody known in the art.

In a further embodiment, antibodies or antibody fragments can be isolated from antibody phage libraries generated using the techniques described in McCafferty et al., Nature, 1990, 348: 552-554. Clackson et al., Nature, 1991, 352: 624-628 and Marks et al., J Mol Biol, 1991, 222: 581-597 describe the isolation of murine and human antibodies, respectively, using phage libraries.

In some embodiments, antibody mimics are useful for this invention. Antibody mimics or “non-antibody binding protein” use non-immunoglobulin protein scaffolds, including adnectins, avimers, single chain polypeptide binding molecules, and antibody-like binding peptidomimetics by using non-immunoglobulin protein scaffolds as alternative protein frameworks for the variable regions of antibodies (U.S. Pat. Nos. 5,260,203; 5,770,380; 6,818,418 and 7,115,396). Other compounds have been developed that target and bind to targets in a manner similar to antibodies. Certain of these “antibody mimics” use non-immunoglobulin protein scaffolds as alternative protein frameworks for the variable regions of antibodies. A methodology for reducing antibodies into smaller peptidomimetics, termed “antibody like binding peptidomimetics” (ABiP) can be used, a methodology for reducing antibodies into smaller peptidomimetics, can also be useful as an alternative to antibodies (Murali et al. Cell Mol Biol., 2003, 49(2):209-216).

In some embodiments, a fusion protein inhibiting or activating certain population of CD4g13 T cells is useful for this invention. Fusion proteins that are single-chain polypeptides including multiple domains termed “avimers” were developed from human extracellular receptor domains by in vitro exon shuffling and phage display and are a class of binding proteins somewhat similar to antibodies in their affinities and specificities for various target molecules (Silverman et al. Nat Biotechnol, 2005, 23: 1556-1561). The resulting multidomain proteins can include multiple independent binding domains that can exhibit improved affinity (in some cases sub-nanomolar) and specificity compared with single-epitope binding proteins. Additional details concerning methods of construction and use of avimers are disclosed, for example, in US Pat. App. Pub. Nos. 20040175756, 20050048512, 20050053973, 20050089932 and 20050221384.

In addition to non-immunoglobulin protein frameworks, antibody properties have also been mimicked in compounds including, but not limited to, RNA molecules and unnatural oligomers (e.g., protease inhibitors, benzodiazepines, purine derivatives and beta-turn mimics) all of which are suitable for use with the present invention. These are aimed to circumvent the limitations of developing antibodies in animals by developing wholly in vitro techniques for designing antibodies of tailored specificity.

In some embodiments, the therapeutic agent that either increases the population of protective CD4g13 T cells (i.e. protective g13Th1 or g13Th2 T cells) or decreases the population of pathological CD4g13 T cells (i.e. pathological g13Th1 or g13Th2 T cells), can be especially useful to prevent or treat an inflammatory or autoimmune disease in a subject. The therapeutic agent of this invention can be administered locally or systemically to a patient.

The specificity of the therapeutic agent of the present invention can be assessed by any method known in the art. The immunoassays that can be used such as, but not limited, to competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Current Protocols in Molecular Biology, (Ausubel et al., eds.), Greene Publishing Associates and Wiley-Interscience, New York, 2002).

### Sample and Method of Measurements

In certain aspects of the present invention, the level of cytokine or biomarker (e.g. gene expression) is determined in a sample obtained from a subject. The sample can be a fluid sample such as a blood sample, preferably containing peripheral blood mononuclear cells (PBMCs), a stool sample, a urine sample, preferably containing infiltrating immune cells, a endometrial biopsy, a sample of synovial fluid, cerebrospinal fluid, or any fluid that is in physiological contact or proximity with the inflammatory region in the subject, or any other body fluid in addition to those recited herein should also be considered to be included in the invention.

Any method known to those in the art can be employed for determining the level of protein (e.g. cytokines) or gene expression (e.g. biomarkers mRNAs) in the sample obtained from the subject. For example, Western blots, arrays or microarrays can be used. Arrays and microarrays are known in the art and consist of a surface to which probes that correspond in sequence to gene products (e.g. mRNAs, polypeptides, fragments thereof etc.) can be specifically hybridized or bound to a known position. To detect at least one protein or mRNA of interest, a hybridization sample is formed by contacting the test sample with at least one ligand, antibody, or nucleic acid probe. As an example, a preferred probe for detecting mRNA is a labeled nucleic acid probe capable of hybridizing to the mRNA. The nucleic acid probe can be, for example, a full-length nucleic acid molecule, or a portion thereof, such as an oligonucleotide of at least 10, 15, or 20 nucleotides in length and sufficient to specifically hybridize under stringent conditions to the appropriate mRNA. The hybridization sample is maintained under conditions which are sufficient to allow specific hybridization of the nucleic acid probe to a mRNA target of interest. Specific hybridization can be performed under high stringency conditions or moderate stringency conditions, as appropriate. In a preferred embodiment, the hybridization conditions for specific hybridization are high stringency. Specific hybridization, if present, is then detected using standard methods. If specific hybridization occurs between the nucleic acid probe and a mRNA in the test sample, the sequence that is present in the nucleic acid probe is also present in the mRNA of the subject. More than one nucleic acid probe can also be used. Hybridization intensity data detected by the scanner are automatically acquired and processed by a software. Raw data is normalized to expression levels using a target intensity of 150. An alternate method to measure mRNA expression profiles of a small number of different genes is by e.g. either classical TaqMan® Gene Expression Assays or TaqMan® Low Density Array—micro fluidic cards (Applied Biosystems) or qPCR system. Other examples of methods that can be employed for determining the level of gene expression is the use of the use of hydrogel particles (e.g. Firefly arrays by BioWorks Inc, Cambridge, Mass. 02139)) or the use of molecular color-coded barcodes and single molecule imaging to detect and count hundreds of unique transcripts in a single reaction such as in the nCounter® system from Nanostring Technology® (Seattle, Wash.). Using this technology, each color-coded barcode is attached to a single target-specific probe corresponding to a gene of interest so that each color-coded barcode represents a single target molecule. Barcodes hybridize directly to the target molecules and can be individually counted without the need for amplification providing very sensitive digital data. After hybridization, the excess probes are removed and the probe/target complexes are aligned and immobilized in the nCounter® Cartridge. The sample Cartridges are placed in the nCounter® Digital Analyzer for data collection and the color codes on the surface of the cartridge are counted and tabulated for each target molecule. The protein level or transcriptional state of a sample may also be measured by other technologies known in the art.

In another aspect, the invention includes a method of assessing the type of immune response in an inflammatory or autoimmune disease in a subject. The method comprising: (a) detecting the level of cytokines IL2, IL13, IL4, IL5, IL17 and IL22 in a sample from the subject; (b) determining the level of gene expression of at least one T cell biomarker selected from the group consisting of: Fam213a, Bmp8, Lrrc32, Cyp11a1, tarm1, Chat2, Chil3, Gpm6b, Bace2, Lag3, Acbd7, Ctse, Hsd17b11, Hrh4, Cd93, Large, Cpa3, Pde8a, Pgr, Nm1, Dapk1, Cyp4f39, Noxred1 and Treml2 in a sample from the subject; (c) comparing the level of the cytokines or the at least one T cell biomarker in the sample from the subject to a baseline level in a control subject not having an inflammatory or autoimmune disease, wherein a higher level of the cytokines or the at least one T cell biomarker in the sample as compared to the level of the cytokines or the at least one T cell biomarker in the control is indicative of a g13Th1 or a g13Th2 T cell response; and, (d) wherein when a g13Th1 or a g13Th2T cell response is indicated, treatment of the inflammatory or autoimmune disease is recommended.

In another aspect, the invention includes a method of detecting a pathological T cell response in an inflammatory or autoimmune disease in a subject. The method comprises (a) determining the presence of a pathological CD4g13 T cell (i.e. pathological g13Th1 or g13Th2) by detecting the level of cytokines IL2, IL13, IL4, IL5 and IFNg in a sample from the subject; (b) determining the level of gene expression of at least one pathological CD4g13 T (i.e. pathological g13Th1 or g13Th2) cell biomarker selected from the group consisting of: Fam213a, Bmp8, Lrrc32, Cyp11a1, tarm1, Chat2, Chil3, Gpm6b, Bace2, Lag3, Acbd7, Ctse, Hsd17b11 Hrh4, Cd93, Large, Cpa3, Pde8a, Pgr, Nm1, Dapk1, Cyp4f39, Noxred1 and Treml2; (c) comparing the level of the cytokines or the at least one pathological CD4g13 T cell biomarker in the sample from the subject to a baseline level in a control subject not having an inflammatory or autoimmune disease, wherein a higher level of the cytokines or the at least one pathological CD4g13 T cell (i.e. pathological g13Th1 or g13Th2) biomarker in the sample as compared to the level of the cytokines or the at least one pathological CD4g13 T (i.e. pathological g13Th1 or g13Th2) cell biomarker in the control is indicative of a pathological T cell response; and, (d) wherein when a pathological T cell response is indicated, treatment of the inflammatory or autoimmune disease is recommended.

In yet another aspect, the invention includes a method of detecting a protective g13Th1 T cell response in an inflammatory or autoimmune disease in a subject. The method comprises (a) determining the presence of a protective g13Th1 T cell by detecting the level of cytokines IL2, IL13, IL17, IL22 and IFNg in a sample from the subject; (b) determining the level of gene expression of at least one g13Th1 T cell biomarker selected from the group consisting of: Cd93, Large, Cpa3, Pde8a, Pgr, Nm1, Dapk1, Cyp4f39, Noxred1 and Treml2; (c) comparing the level of the cytokines or the at least one g13Th1 cell biomarker in the sample from the subject to a baseline level in a control subject not having an inflammatory or autoimmune disease, wherein a higher level of the cytokines or the at least one g13Th1 T cell biomarker in the sample as compared to the level of the cytokines and/or to the at least one g13Th1 T cell biomarker in the control is indicative of a protective g13Th1 T cell response; and, (d) wherein when a treatment of the inflammatory or autoimmune disease is recommended. The treatment can include administering an agent to combat the disease, or administering a vaccine that protects against the disease.

In yet another aspect, the invention includes a method of detecting a protective g13Th2 T cell response in an inflammatory or autoimmune disease in a subject. The method comprises (a) determining the presence of a protective g13Th2 T cell by detecting the level of cytokines IL2, IL13, IL4, IL5 and IFNg in a sample from the subject; (b) determining the level of gene expression of at least one g13Th2 T cell biomarker selected from the group consisting of: Fam213a, Bmp8, Lrrc32, Cyp11a1, tarm1, Chat2, Chil3, Gpm6b, Bace2, Lag3, Acbd7, Ctse, Hsd17b11 and Hrh4; (c) comparing the level of the cytokines or the at least one g13Th2 cell biomarker in the sample from the subject to a baseline level in a control subject not having an inflammatory or autoimmune disease, wherein a higher level of the cytokines or the at least one g13Th2 T cell biomarker in the sample as compared to the level of the cytokines and/or to the at least one g13Th2 T cell biomarker in the control is indicative of a protective g13Th2 T cell response; and, (d) wherein when a protective T cell response is indicated, a treatment of the inflammatory or autoimmune disease is recommended. The treatment can include administering an agent to combat the disease, or administering a vaccine that protects against the disease.

In yet another aspect, the invention includes a method of assessing a response to a therapeutic agent in a subject, the method comprises (a) administering an effective amount of the therapeutic agent to the subject(b) determining the presence of a protective g13Th1 T cell by detecting the level of cytokines IL2, IL13, IL17, IL22 and IFNg in a sample from the subject; (c) determining the level of gene expression of at least one g13Th1 T cell biomarker selected from the group consisting of: Cd93, Large, Cpa3, Pde8a, Pgr, Nm1, Dapk1, Cyp4f39, Noxred1 and Treml2; (d) comparing the level of the cytokines or the at least one g13Th1 T cell biomarker in the sample from the subject to a baseline level in a control subject not having an inflammatory or autoimmune disease, wherein a higher level of the cytokines or the at least one protective g13Th1 T cell biomarker in the sample as compared to the level of the cytokines and/or to the at least one g13Th1 T cell biomarker in the control is indicative of a protective therapeutic agent generated T cell response.

In yet another aspect, the invention includes a method of assessing a response to a therapeutic agent in a subject, the method comprises (a) administering an effective amount of therapeutic agent to the subject; (b) determining the presence of a protective g13Th2 T cell by detecting the level of cytokines IL2, IL13, IL4, IL5 and IFNg in a sample from the subject; (c) determining the level of gene expression of at least one g13Th2 T cell biomarker selected from the group consisting of: Fam213a, Bmp8, Lrrc32, Cyp11a1, tarm1, Chat2, Chil3, Gpm6b, Bace2, Lag3, Acbd7, Ctse, Hsd17b11 and Hrh4; (d) comparing the level of the cytokines or the at least one g13Th2 T cell biomarker in the sample from the subject to a baseline level in a control subject not having an inflammatory or autoimmune disease, wherein a higher level of the cytokines or the at least one g13Th2 T cell biomarker in the sample as compared to the level of the cytokines and/or to the at least one g13Th2 T cell biomarker in the control is indicative of a protective therapeutic agent-generated T cell response.

### Control

The method of the invention includes comparing the level of the CD4g13 T cells' cytokines or biomarkers in a biological sample from a subject to a control amount (i.e. the reference).

As used herein, the terms “control,” or “reference” are used interchangeably, and refer to a value that is used as a standard of comparison (e.g., level of CD4 T cells in a healthy subject).

As used herein, “Baseline level” includes the particular protein or gene expression level of a healthy subject or a subject without any inflammatory or autoimmune disease.

In one embodiment, the baseline level of a protein (cytokine) or gene expression (biomarker) includes the protein or gene expression level of a healthy subject or a subject without any inflammatory or autoimmune disease. Preferably, the healthy subject is a subject of similar age, gender and race and has never been diagnosed with any type of severe disease particularly any inflammatory or autoimmune disease.

The baseline level of protein or gene expression can be a number on paper or the baseline level of gene expression from a control sample of a healthy subject or a subject without any inflammatory or autoimmune disease.

In another embodiment, the baseline level of protein or gene expression can be a number on paper or a value for CD4g13 T cells (such as a value for protective and/or pathological g13Th1 or g13Th2 T cells) that is accepted in the art. This reference value can be baseline value calculated for a group of subjects based on the average or mean values of protein level (e.g. IL-4 and IL-5 or IL-17 and IL-22) or gene expression (e.g. Cd93, Large, Cpa3, Pde8a, Pgr, Nm1, Dapk1, Cyp4f39, Noxred1 and Treml2 or Fam213a, Bmp8, Lrrc32, Cyp11a1, tarm1, Chat2, Chil3, Gpm6b, Bace2, Lag3, Acbd7, Ctse, Hsd17b11 and Hrh4) by applying standard statistically methods.

In one embodiment, the expression level is determined by a method selected from the group consisting of detecting mRNA of the gene, detecting a protein encoded by the gene, and detecting a biological activity of the protein encoded by the gene.

### Pharmaceutical Compositions and Formulations.

The invention further includes a pharmaceutical composition comprising a therapeutic agent that increases the population of protective CD4g13, g13Th1 or g13Th2 T cells, for use in the methods of the invention.

Such a pharmaceutical composition is in a form suitable for administration to a subject, or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

In an embodiment, the pharmaceutical compositions useful for practicing the method of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for inhalational, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In one embodiment, the compositions are formulated using one or more pharmaceutically acceptable excipients or carriers. In one embodiment, the pharmaceutical compositions comprise a therapeutic agent that increases or decreases the population of CD4g13 T cells and a pharmaceutical acceptable carrier. Pharmaceutically acceptable carriers, which are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences, 1991, Mack Publication Co., New Jersey.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

### Administration/Dosing

The regimen of administration may affect what constitutes an effective amount. For example, the therapeutic formulations may be administered to the patient either prior to or after a surgical intervention related to cancer, or shortly after the patient was diagnosed with cancer. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, preferably a mammal, more preferably a human, may be carried out using known procedures, at dosages and for periods of time effective to treat cancer in the patient. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 0.01 and 50 mg/kg of body weight/per day. One of ordinary skill in the art would be able to study the relevant factors and make the determination regarding the effective amount of the therapeutic compound without undue experimentation.

The compound can be administered to an animal as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon any number of factors, such as, but not limited to, the type and severity of the disease being treated, and the type and age of the animal. Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient. A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of cancer in a patient.

### Routes of Administration

One skilled in the art will recognize that although more than one route can be used for administration, a particular route can provide a more immediate and more effective reaction than another route.

Routes of administration of any of the compositions of the invention include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration. Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Controlled Release Formulations and Drug Delivery Systems

Controlled- or sustained-release formulations of a pharmaceutical composition of the invention may be made using conventional technology. In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, micro-particles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gelcaps, and caplets, which are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

### Immune Response Stimulation

In one embodiment, the invention comprises a method for stimulating a protective CD4g13 T cell-mediated immune response, g13Th1 or g13Th2, to a cell population or a local tissue or organ in a subject in need thereof. The method comprises administering to the subject an effective amount a therapeutic agent that increases the population of protective CD4g13 T cells and a pharmaceutical acceptable carrier.

The activation T lymphocytes (T cells) and its use within immunotherapy for the treatment of cancer and infectious diseases, is well known in the art (Melief et al., Immunol. Rev., 1995, 145:167-177; Riddell et al., Annu. Rev. Immunol., 1995, 13:545-586). As disclosed in the current invention, stimulation of protective g13Th1 T cells leads to an increase in the level of cytokines IL2, IL13, IL17, IL22 and IFNg. In some embodiments, the level of cytokines IL17 and IL22 is increased. In other embodiments, stimulation of protective g13Th1 cells can be assessed by measuring specific g13Th1 T cells biomarkers such as, but are not limited to, Cd93, Large, Cpa3, Pde8a, Pgr, Nm1, Dapk1, Cyp4f39, Noxred1 and Treml2. The measurement in a sample of level of at least one of these cytokines or biomarkers can be used to assess protective g13Th1 T cells activation as presented herein the Examples section. Sorting of T cells, or generally any cells of the present invention, can be carried out using any of a variety of commercially available cell sorters, including, but not limited to, MoFlo sorter (DakoCytomation, Fort Collins, Colo.), FACSAria™ FACSArray™, FACSVantage™, BD™ LSR II, and FACSCalibur™ (BD Biosciences, San Jose, Calif.).

As disclosed herein, stimulation of protective g13Th2 T cells leads to an increase in the level of cytokines IL2, IL13, IL4, IL5 and IFNg. In some embodiments, the level of cytokines IL4 and IL5 is increased. In other embodiments, stimulation of protective g13Th2 cells can be assessed by measuring specific g13Th2 T cells biomarkers such as, but are not limited to, Fam213a, Bmp8, Lrrc32, Cyp11a1, tarm1, Chat2, Chil3, Gpm6b, Bace2, Lag3, Acbd7, Ctse, Hsd17b11 and Hrh4. The measurement in a sample of level of at least one of these cytokines or biomarkers can be used to assess protective g13Th2 T cells activation as presented herein the Examples section. Sorting of T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

### Kits

The invention includes a set of antibodies, polypeptides, nucleic acids, or mRNAs either labeled (e.g., fluorescer, quencher, etc.) or unlabeled, that are useful for the detection of a protective or a pathological CD4g13 (i.e. g13Th1 or g13Th2) T cell response in a subject.

In certain embodiments, a kit is provided. Commercially available kits for use in these methods are, in view of this specification, known to those of skill in the art. In general, kits comprise a detection reagent that is suitable for detecting the presence of a polypeptide or nucleic acid, or mRNA of interest.

In another embodiment, there is a panel of probe sets or antibodies. In some embodiments, the panel of probe sets is designed to detect at least one biomarker and provide information about the type of T cell response in an inflammatory or autoimmune disease in a subject (i.e. a protective or pathological T cell response). In one embodiment, the at least one biomarker is selected from the group consisting of Cd93, Large, Cpa3, Pde8a, Pgr, Nm1, Dapk1, Cyp4f39, Noxred1 and Treml2 and is used to diagnose a g13Th1 T cell response. In another embodiment, the at least one biomarker is selected from the group consisting of Fam213a, Bmp8, Lrrc32, Cyp11a1, tarm1, Chat2, Chil3, Gpm6b, Bace2, Lag3, Acbd7, Ctse, Hsd17b11 and Hrh4 and is used to diagnosed a g13Th2 T cell response. Probe sets are particularly useful because they are smaller and cheaper than probe sets that are intended to detect as many peptides as possible in a particular genome. In the present invention, the probe sets are targeted at the detection of polypeptides that are informative about cancer genes. Probe sets may also comprise a large or small number of probes that detect peptides that are not informative about cancer. Such probes are useful as controls and for normalization (e.g., spiked-in markers). Probe sets may be a dry mixture or a mixture in solution. In some embodiments, probe sets can be affixed to a solid substrate to form an array of probes. The probes may be antibodies, or nucleic acids (e.g., DNA, RNA, chemically modified forms of DNA and RNA), LNAs (Locked nucleic acids), or PNAs (Peptide nucleic acids), or any other polymeric compound capable of specifically interacting with the peptides or nucleic acid sequences of interest.

It is contemplated that kits can be designed for isolating and/or detecting peptides (e.g. CD4g13 T cells biomarkers, immune activators or apoptotic proteins) or nucleic acid sequences in essentially any sample (e.g., blood, cells, tissue, stool etc.), and a wide variety of reagents and methods are, in view of this specification, known in the art.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The Materials and Methods used in the performance of the experiments disclosed herein are now described.

### Mice

4-5 week old female C57BL/6 mice were purchased from Harlan Labs (Indianapolis, Ind.) and Jackson Labs (Bar Harbor, Mass.). Mice were housed in Indiana University Purdue University-Indianapolis (IUPUI) and Yale University specific-pathogen-free facilities (SPF). The Institutional Animal Care and Utilization Committees at Indiana University, Yale University, and University of British Columbia approved all experimental protocols.

### Cells and Bacteria

McCoy fibroblasts were cultured as previously described (Jayarapu et al., 2009, Infect Immun 77:4469-4479). Mycoplasma-free Chlamydia muridarum (Nigg), previously known as C. trachomatis strain mouse pneumonitis (MoPn) (Nigg) was grown in McCoy cells as previously described (McCully et al., 2012, Blood 120:4591-4598). Soluble Chlamydia antigen (infected cell lysate depleted of EB by centrifugation) was prepared as previously described (Johnson et al., 2014, Immunology 142:248-257), aliquoted and stored at −80° C.

### Chlamydia-Specific CD4 T Cells

Conventional multifunction Chlamydia-specific Th1 clone 4uvmo-3 was previously described (Jayarapu et al., 2009, Infect Immun 77:4469-4479). For the new B-cell APC-derived T cells C57BL/6 mice were treated with 2.5 mg of medroxyprogesterone (Pfizer) delivered subcutaneously, then infected 7 days later with 5×10⁴ IFU C. muridarum. Mice that cleared infection, >6 weeks post-infection, were used as the source of immune B and T cells. Initial Chlamydia-specific immune-B-cell-derived polyclonal T cell populations and clones were derived as follows. Splenocytes were harvested from immune mice. Immune B cells were purified from a portion of those splenocytes by “untouched” magnetic bead separation (Miltenyi Biotech). Immune B cells were pulsed with UV-MoPn (3.5×10⁶ IFU equivalents per 7.5×10⁵ B cells suspended at 7.5×10⁶/ml; ˜5 IFU/cell) or soluble antigen (7.5 ul per 7.5×10⁵ B cells suspended at 7.5×10⁶/ml) for 1 h at 37° C. Antigen-pulsed immune B cells were transferred to 7.5 cc of “RPMI complete media”, pelleted, media containing antigen removed, then washed two more times with 7.5 cc of media (˜400,000-fold) to eliminate all non-cell bound or internalized Chlamydia antigen; the purpose of extended washing was to ensure that antigen presentation was limited to immune B cells. Primary stimulation wells were setup with 2.5×10⁶ immune splenocytes plus 7.5×10⁵ antigen-pulsed immune B cells in 0.75 ml “RPMI complete media” supplemented with recombinant cytokines and conditioned media as previously described (Johnson et al., 2014, Immunology 142:248-257); later T cell derivations during the course of the project included addition of 5 ng/ml recombinant murine TGFβ1 to the media. Limiting dilution cloning was done in media supplemented with recombinant cytokines/conditioned media with 10-20 ng/ml TGFβ1. Re-stimulation/maintenance of T cell clones was done weekly in 48-well plates by adding 100-200 k T cell clone cells to 1.5×10⁶ irradiated naive splenocytes and 7.5×10⁵ irradiated relevant-antigen-pulsed/washed immune B cells as feeders in media supplemented with recombinant cytokines/conditioned media including 2.5-10 ng/ml TGFβ1. Recombinant mouse cytokines were purchased from the same vendor (R&D Systems; Minneapolis, Minn.) except for TGFP1 (Ebioscience, San Diego, Calif.).

### Flow Cytometry and Intracellular Cytokine Staining

For B cell staining For B cell staining, single cell suspensions of genital tracts pooled from four mice per experimental group were surface stained using anti-mouse B220 (RA3-6B2 coupled to FITC, BD Pharmingen) as well as with the viability dye, aqua fluorescent reactive dye (L34957; Molecular Probes), followed by intracellular staining using anti-mouse CD79a (24C2.5 coupled to eFluor660, eBioscience). The experiment was repeated 1 or 2 times for individual experimental groups. T cell surface phenotypes were determined using antibodies to CD4 (GK1.5 coupled to phycoerythrin), CD8a (53-6.7 coupled to FITC). T cells were stained for 20 min at 4° C. with 1 ug per 1 million T cells in RPMI CM with 10% FBS, fixed with 1% paraformaldehyde and analyzed by flow cytometry (BD Facscalibur or LSRII). For intracellular staining for IL-13(ebio13A coupled to PE) and IFN-g (XMG1.2 coupled to APC), T cells were activated for 5 h in cocktail of phorbol 12-myristate 13-acetate (PMA), ionomycin, brefeldin A and monensin (cell stimulation cocktail, Ebioscience), stained for CD8a, then fixed and permeabilized (fix/perm buffer, Ebioscience), stained for IL-13(PE)/IFN-g (APC) or control antibody (eBRG1-PE)/IFN-g (APC) in presence of 2 mg/ml donkey IgG (Jackson Immunoresearch) for 30 min at room temp, washed, suspended in 1% paraformaldehyde and analyzed. All the T cell populations are >90% CD4 T cells; negative staining based on CD8a was chosen because PMA/ionomycin activation resulted in shedding of cell surface CD4 and diminished CD4 staining; CD8a staining was not affected.

### Cytokine ELISAs and Signaling Reagents

2.5×10⁴ ficoll-hypaque purified T cell clones (5×10⁴ purified T cells for IL-4 determination) cultured overnight in RPMI media with 3 ng/ml IL-7 were activated in 96-well tissue culture plates by immobilized anti-CD3 monoclonal antibody 145-2c11, 0.5 ng/ml in PBS overnight at 4° C. (washed once), in RPMI media containing 1 ng/ml recombinant murine IL-7(R&D Systems, Minneapolis, Minn.) for 20 h. Relative levels of IL-2, interferon-gamma (IFN-g), IL-13, IL-10, TNFα, IL-17, IL-22, IL-4 and IL-5 in culture supernatants were determined by ELISA using capture and biotinylated monoclonal antibody pairs with recombinant murine standards according to the manufacturer's protocols. IL-2: JES6-1A12/Jes6-5H4; IFN-g ELISA: XMG1.2; (Pierce-Thermofisher; Rockford Ill.); IL-13 ELISA eBio13A/eBio1316H (Ebioscience); IL-10: Jess-16E3/Jess-2A5; TNFα: TN3-19.12/rabbit anti-mouse/rat polyclonal (BD Biosciences); IL-17: 17CK15A5/17B7 (Ebioscience); IL-22 polyclonal 5164 (Biolegend); IL-5: TRFK5/TRFK4. IL-4: 11b11/BVD6-24g2) (Ebioscience). Detection was accomplished with Streptavidin-HRP (BD Biosciences) and TMB substrate (Sigma Chemical Co). Cyclosporine A was purchased from Sigma and dissolved in ethanol. CrTh2 inhibitors I ((4-Chloro-2-((2-methyl-5 (propyl sulfonyl)phenyl)ethynyl)phenoxy)acetic Acid) and II ((R)-(5-Chloro-1′-(5-chloro-2-fluorobenzyl)-2,2′,5′-trioxospiro(indole-3,3″-pyrrolidin)-1(2H)-yl)acetic acid) were purchased from EMD Millipore (Temecula, Calif.) and dissolved in DMSO.

### Redirected Lysis

Redirected lysis was performed as described by Leo et al. (Leo et al., 1986, J Immunol 137:3874-3880). A total of 10,000 P815 cells (ATCC TIB-64, American Type Culture Collection, Manassas, Va.), a mastocytoma cell line expressing FcRs, was incubated with 10,000 CD4 T cells in the presence of 0.5\hat{ }g/ml anti-CD3e (clone 145-2c11, NA/LE, BD Biosciences, San Jose, Calif.) in 96-well v-bottom plates spun 1 min at 300×g then incubated for 4 h. Killing was quantified using a nonradioactive cytotoxicity assay measuring release of lactate dehydrogenase activity in culture supernatant (cyto 96, Promega, Madison, Wis.) following the manufacturer's protocol. The lysis assays were done using RPMI CM with 1% heat-inactivated serum (68° C. for 30 min to inactivate lactate dehydrogenase activity present in FBS). % specific lysis calculated as: [(experimental release T cells+P815+anti-CD3)−(spontaneous release T cells+P815 without antibody)/(maximal release triton X-100 treatment of P815)]×100.

### Adoptive Transfer and Genital Tract Infections

T cells were purified with ficoll-hypaque (histopaque 1083; SigmaChemical Co, St. Louis, Mo.) on day 5 of the culture cycle and maintained in RPMI complete media with 3 ng/ml murine recombinant IL-7 for 2 days prior to adoptive transfer. One week prior to infection mice were treated with 2.5 mg of medroxyprogesterone (Pfizer) delivered subcutaneously. Six days later 1×10⁶ T cell clone cells were adoptively transferred via retro-orbital injection into fully anesthetized mice; controls were injected with an equivalent volume of phosphate buffered saline (PBS). The day following adoptive transfer lightly anesthetized mice were infected vaginally with 5×10⁴ inclusion forming units (IFU) of C. muridarum in 10 ul of SPG buffer. Mice were serially swabbed through day 30 post infection and IFU determined on McCoy cells to quantify bacterial shedding. On day 56 post infection the mice were killed and genital tracts scored for pathology as previously described (Johnson et al., 2012, J Immunol 188:1896-1904). Briefly, each mouse genital tract has 2 uteri and 2 oviducts; one point is assigned for macroscopic (visible) thinning-dilatation of each site for a maximum score of 4 per mouse. Scoring is done in situ; the genital tracts are then excised and photographed for a digital record (qualitative data). The adoptive transfer experiments were aggregated for Chi-squared analysis.

### Gene Expression Micro Array Analysis

For the “resting” phenotype micro arrays Chlamydia-specific CD4 T cell clones 4uvmo-3, BT12-7, BT12-17, sBT13-11, sBT13-7 and sBT16-8 were purified by ficoll-hypaque at the end of their usual 7-day culture cycle, and then maintained in RPMI complete media with 3 ng/ml IL-7 for 48 h without antigen stimulation. Total RNA was isolated from each T cell clone using a protocol that included a genomic DNA removal step (G-eliminator; RNeasy; Qiagen, Valencia, Calif.). RNA isolation under these culture conditions was repeated 4 times (independent experiments) for each clone to minimize false discovery. With assistance from The Indiana University Center for Medical Genomics, gene expression patterns were analyzed using the Affymetrix Clariom S mouse arrays that analyzes >20,000 well-annotated genes. Samples were labeled using the standard Affymetrix protocol for the Affymetrix WT Plus kit using 100 ng of total RNA. Individual labeled samples were hybridized to the Mouse Clariom S GeneChips® for 17 hours then washed, stained and scanned with the standard protocol using Affymetrix GeneChip® Command Console Software (AGCC) to generate data (CEL files). Arrays were visually scanned for abnormalities or defects. CEL files were imported into Partek Genomics Suite (Partek, Inc., St. Louis, Mo.). The microarray data presented here is available in the Gene Expression Omnibus database (www.ncbi.nlm.nih.gov/geo) under accession number GSE104743.

### Western Blots

Polyclonal rabbit antiserum specific for Eomes (ThermoFisher Scientific cat #720202), Tbet/Gata3/Fhl2 (Proteintech cat #s 13700-1-AP/10417-1-AP/21619-1-AP), and (HRP-coupled monoclonal antibody to beta actin (Sigma Aldrich cat #A3854) were obtained from commercial vendors. 10 lag of whole cell lysate protein was run on 4-12% gradient gels, transferred to nitrocellulose using a dry blotting system (iBlot; ThermoFisher). Membranes were rinsed in TBST and blocked with 5% non-fat milk in TBST. Rabbit antisera were detected with a rabbit-specific chemiluminescent kit (ThermoFisher cat #WB7106). Detection was a commercial chemiluminescent substrate (ThermoFisher cat #34080).

### Statistical Methods

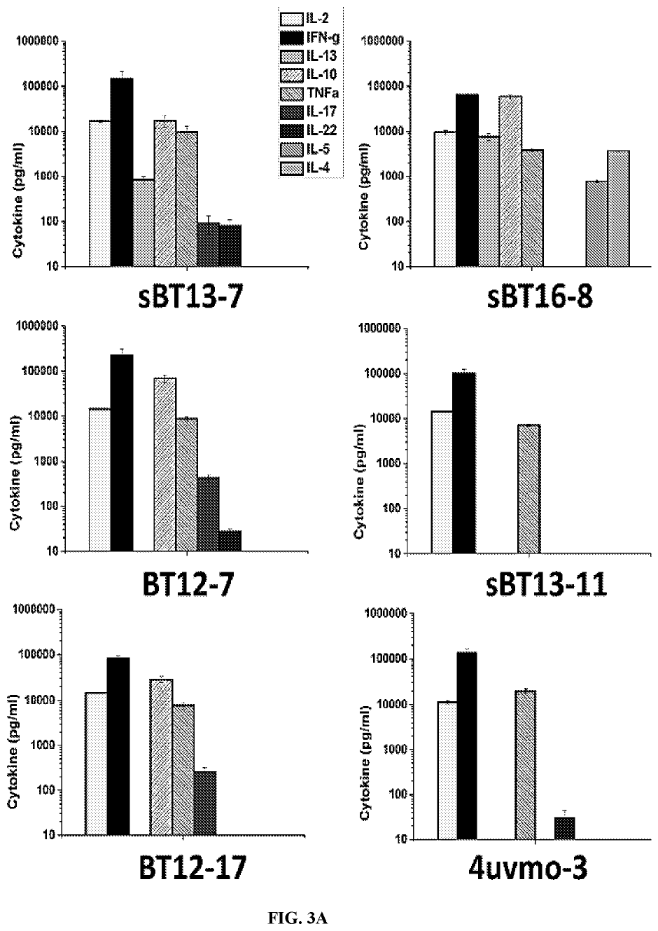

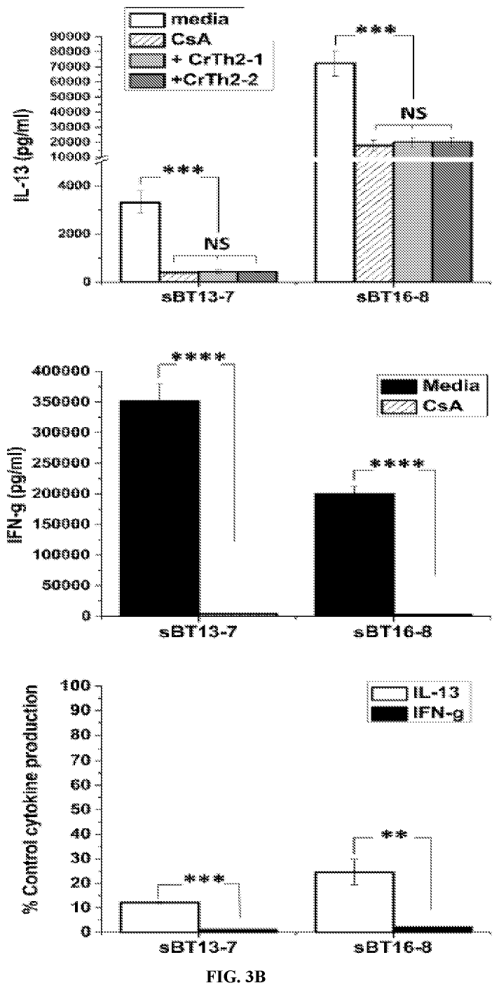

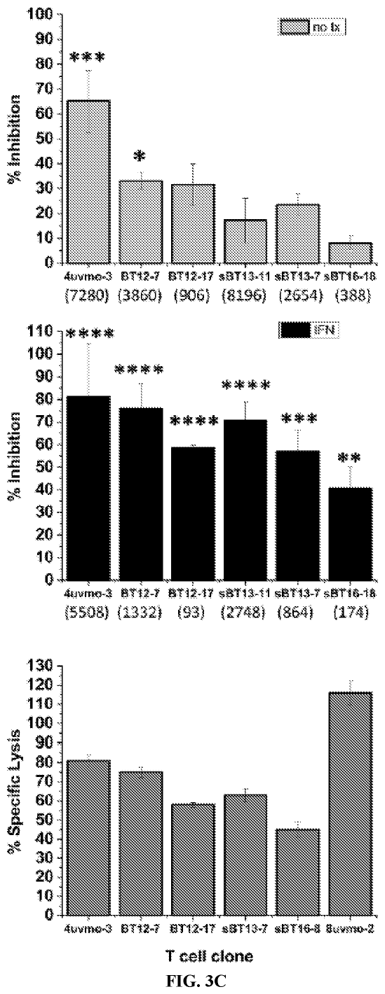

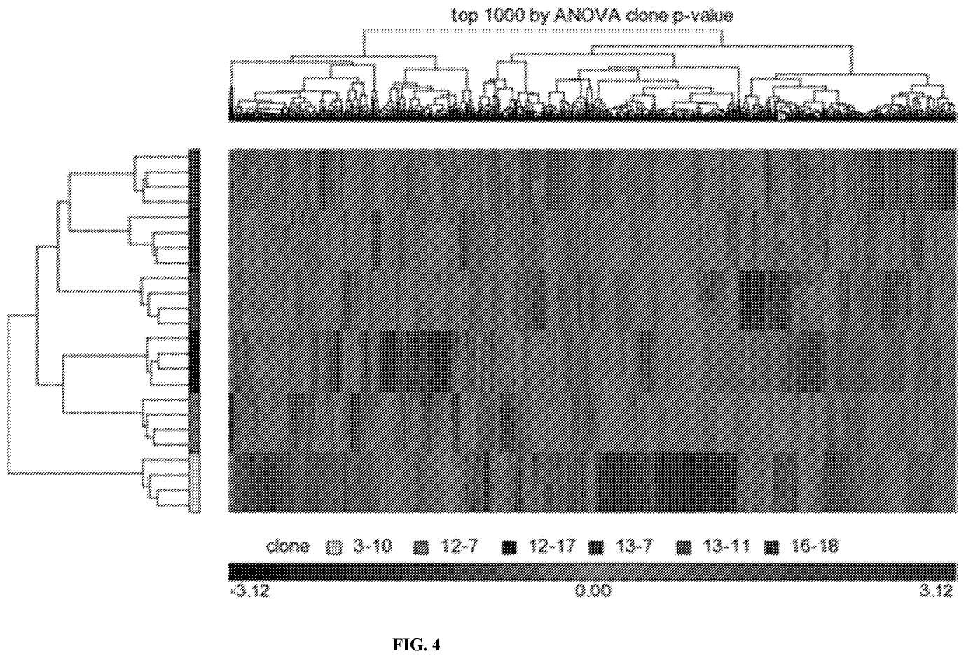

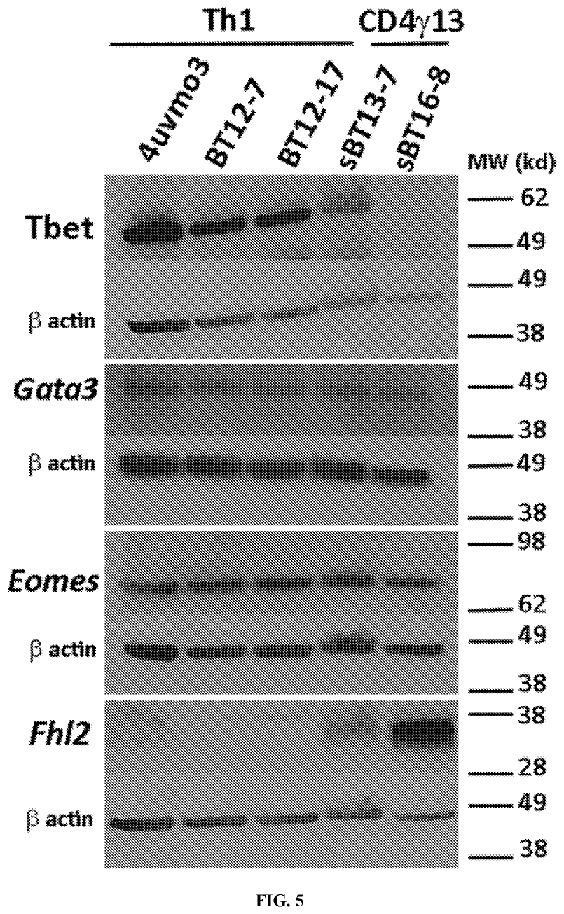

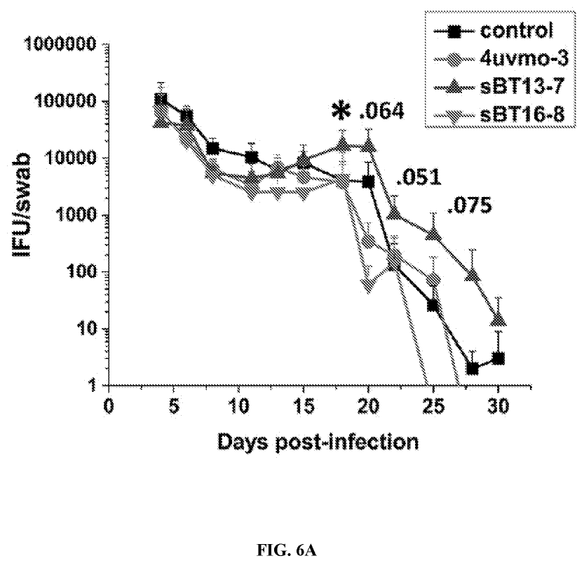

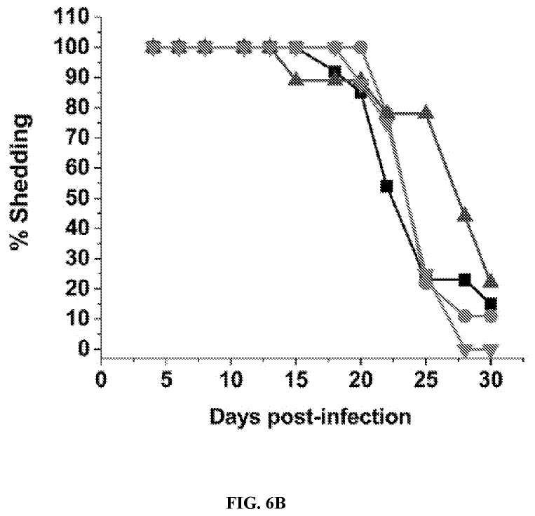

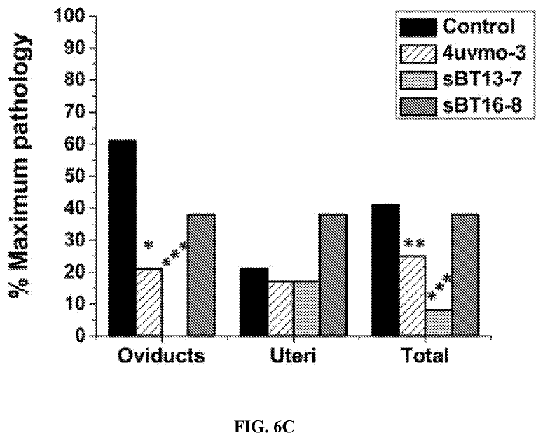

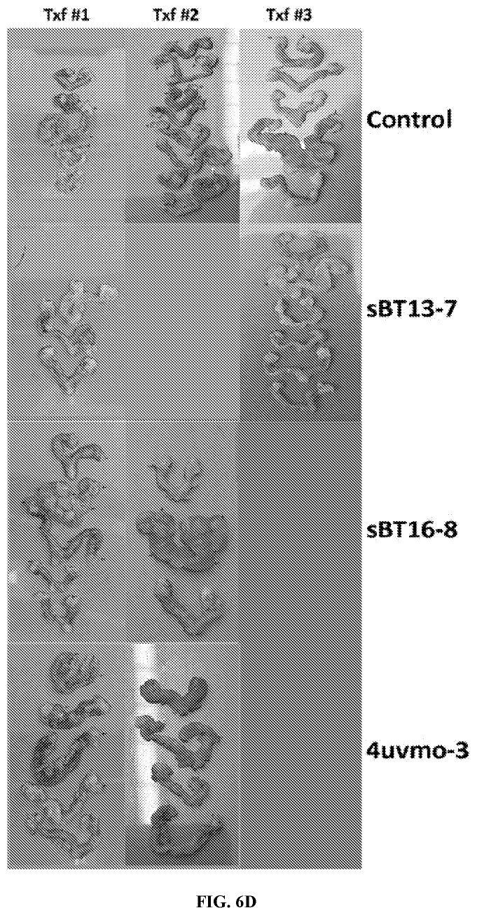

As indicated in each figure legend aggregated data was analyzed with two sample Students t-test using Origin 8.0 software. Exceptions were FIG. 3B and FIG. 4 (ANOVA) and FIGS. 6A-6D (Dunnett's test); performed using R software, and Chi-squared analysis of FIG. 6C.

The results of the experiments are now described.

### Example 1: Plasma Cells in the Genital Tract

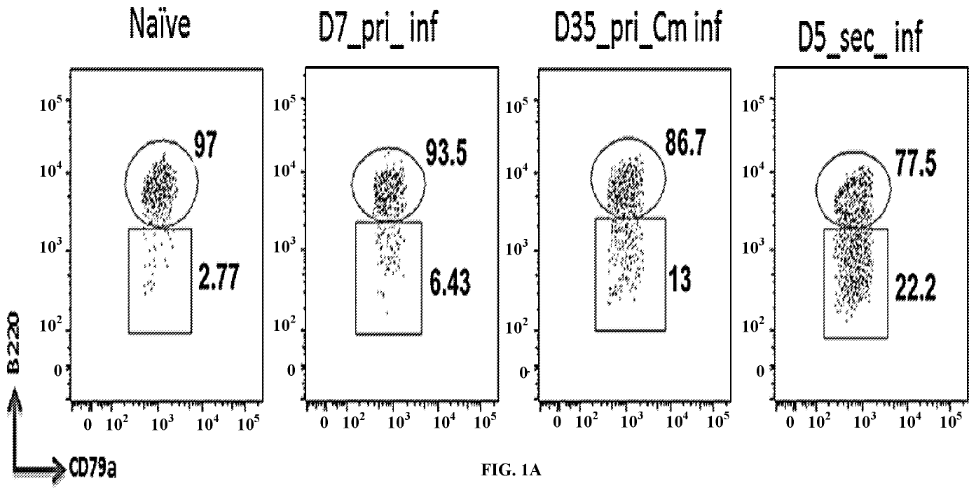

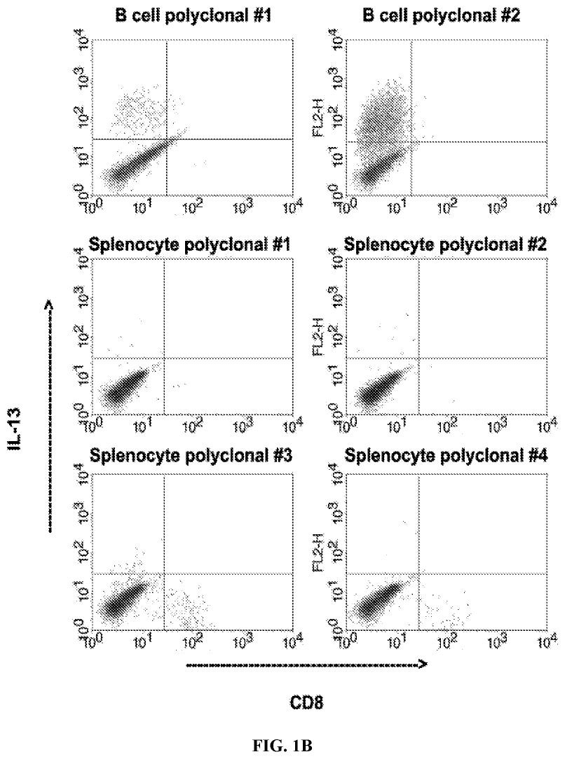

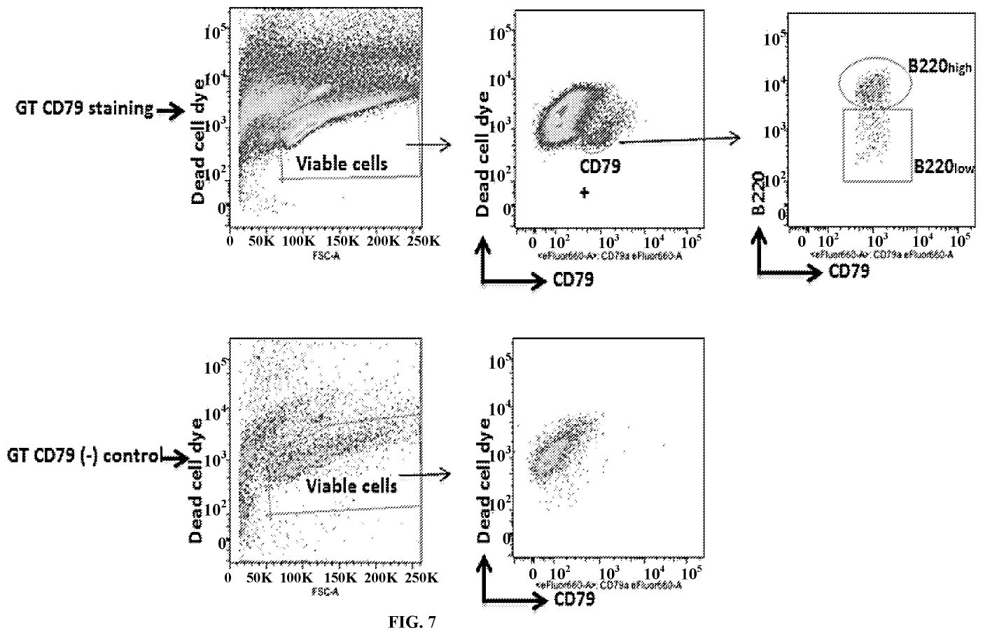

Chlamydia pathogenesis viewed through the lens of tissue resident immunity rather than cytokine polarization (Th1/2/17), showed, in human studies, that B lymphocytes and plasma B cells are prominent in Chlamydia infection-associated memory lymphocyte clusters (c-MLC) (Johnson et al., 2016, Infect Immun 84:868-873). B lymphocyte data in the C. muridarum mouse model is inconclusive due to utilization of B220 staining, a marker down regulated when B lymphocytes transition to immune plasma B cells. To address the discrepancy between human and mouse data, B cell dynamics was determined herein in the genital tract over the course of a C. muridarum infection, gating on CD79a and measuring the relative levels of B lymphocytes (B220 high) and plasma B cells (B220 low) (FIG. 1A; gating strategy in FIG. 7). Gating on CD79a allows detection of plasma B cells that do not express B220 (Dustin et al., 1995, J Immunol 154:4936-4949). In naive mice very few plasma cells reside in the genital tract. During the course of a C. muridarum genital tract infection the 95 percentage of plasma cells increases from a baseline of 3% to 13%, with a further expansion to 22% during re-challenge infections. The results in FIG. 1A show that plasma B cells are nearly absent in a naive genital tract, and expand as demonstrable immunity develops over the course of a primary infection.

### Example 2: Immune B Cells as Antigen Presenting Cells

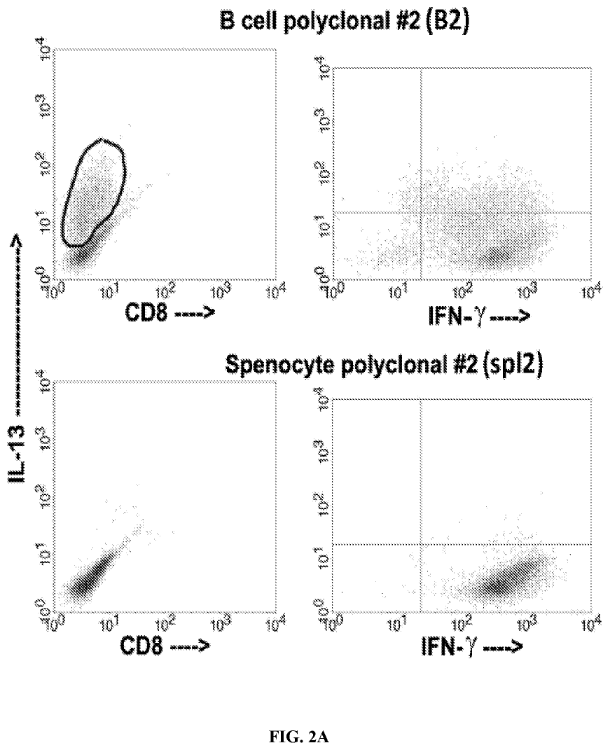

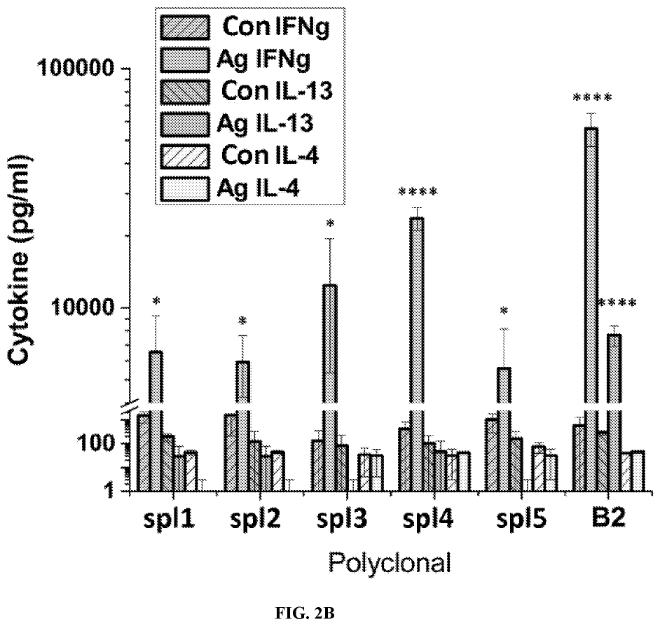

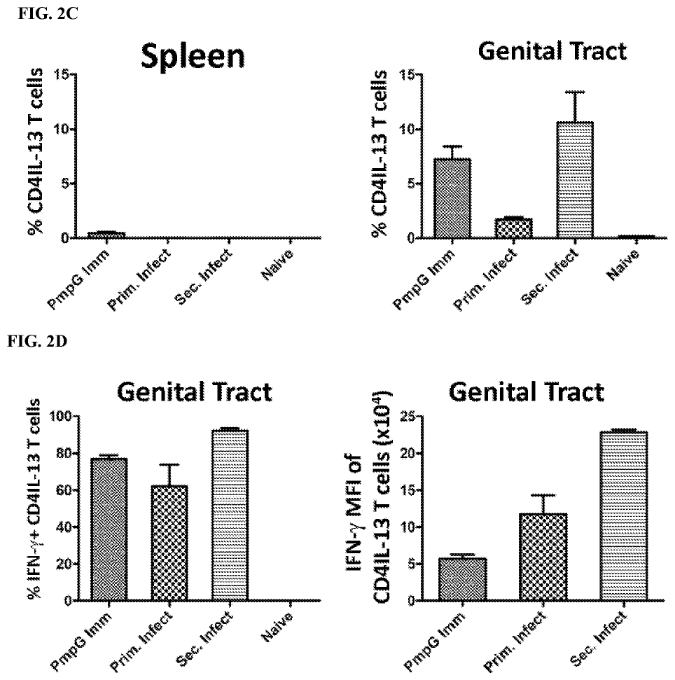

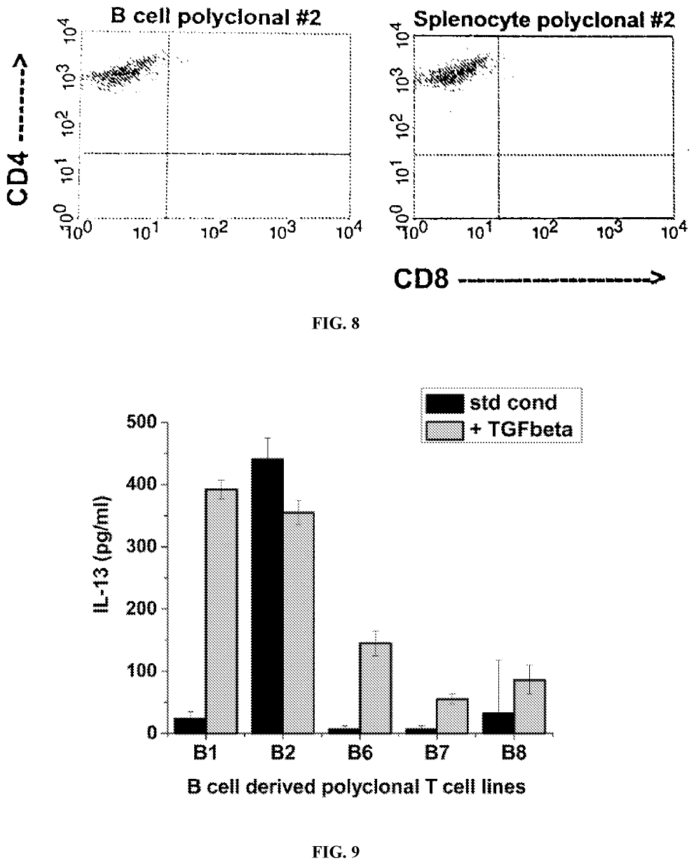

B cells from immunized mice bearing endogenous immunoglobulins (single specificity), and a sampling of serum IgG (multiple specificities) bound to their cell surfaces via Fc receptors can activate T cells at cognate antigen concentrations 1000-fold lower than do naive B cells (Rock et al., 1984, J Exp Med 160:1102-1113); i.e. are 1000× more potent as APC. B cells purified from mice that previously self-cleared C. muridarum genital tract infections (immune mice) are referred herein and thereafter as “immune B cells”. The nature of Chlamydia-specific T cells recovered from immune mice using immune B cells as APC was investigated; utilizing splenocytes rather than genital tract lymphocytes based limited cell numbers in genital tract tissue and the need to develop untested methodologies. Splenic B cells were purified from an immune mouse, pulsed them with UV-inactivated Chlamydia muridarum (uvMoPn), then washed them extensively (400,000-fold) to remove all antigen not bound to or internalized by the immune B cells, thereby limiting antigen presentation to B cells. Antigen-pulsed/washed B cells (immune-B-cell-APC) were co-cultured with splenocytes from the same immune mouse in two primary wells to expand T cells. In parallel, for comparison, from the same mouse, T cells were expanded in two primary wells using uvMoPn and unfractionated immune splenocytes (immune-splenocyte-APC) (Johnson et al., 2014, Immunology 142:248-257). At passage #3, a flow cytometry was performed to determine relative CD4/CD8 numbers; >95% of the resulting T cells populations from the expansions based on immune-B-cell-APC and the expansions based on immune-splenocyte-APC were CD4 T cells (FIG. 8). Though none of the current Chlamydia-specific CD4 T cell murine studies had evidence for a CD4 IL-13 T cell response, IL-13 was of particular interest herein because of its association with immune protection and pathology. Upon activation the two immune-B-cell-APC-derived polyclonal T cell lines produced IL-13 while the two immune-splenocyte-APC-derived T cell populations did not. The two immune-B-cell-APC-derived T cell lines were propagated and immune-splenocyte-APC-derived polyclonal T cell lines were generated from four additional mice that previously cleared C. muridarum genital tract infections. Two immune-B-cell-APC-derived and four immune-splenocyte-APC-derived polyclonal T cell lines were activated with PMA/ionomycin and stained CD8 versus IL-13 (negative CD8 staining was intentionally used to identify CD4 T cells in this assay; see material and methods) (FIG. 1B). The immune-B-cell-APC-derived polyclonal lines included a subset of CD4 T cells producing IL-13; none of the four immune-splenocyte-APC-derived polyclonal T cell lines had a CD4IL-13 T cell subset. One immune-B-cell-APC-derived T cell line and one immune-splenocyte-APC-derived T cell line from the same mouse were activated, and stained intracellularly for IFN-g and IL-13 (FIG. 2A). The immune-splenocyte-APC-derived CD4 T cell line did not stain for IL-13; all the T cells in the immune-B-cell-APC-derived T cell line that produced IL-13 also produced IFN-g. An immune-B-cell-APC-derived T cell line (B2) and five immune-splenocyte-APC-derived T cell lines (sp11-5) were activated with purified uvMoPn-pulsed immune B cells and measured IFN-g, IL-13, and IL-4 in culture supernatant (FIG. 2B). All the polyclonal T cell lines were Chlamydia-specific; when activated by antigen-pulsed B cells they all produced IFN-g; only the immune-B-cell-APC-derived T cell line B2 produced IL-13; none produced IL-4. The results showed that IL-13 production in immune-B-cell-APC-derived T cell lines faded with increasing passage number, that durable IL-13 production by immune-B-cell-APC-derived polyclonal T cell lines required addition of TGFβ1 to the media (e.g. FIG. 9), and that the TGFβ1 effect was specific to T cell lines derived with immune-B-cell-APC, i.e. that addition of TGFβ1 to immune-splenocyte-APC-derived T cell lines did not result in IL-13 production.

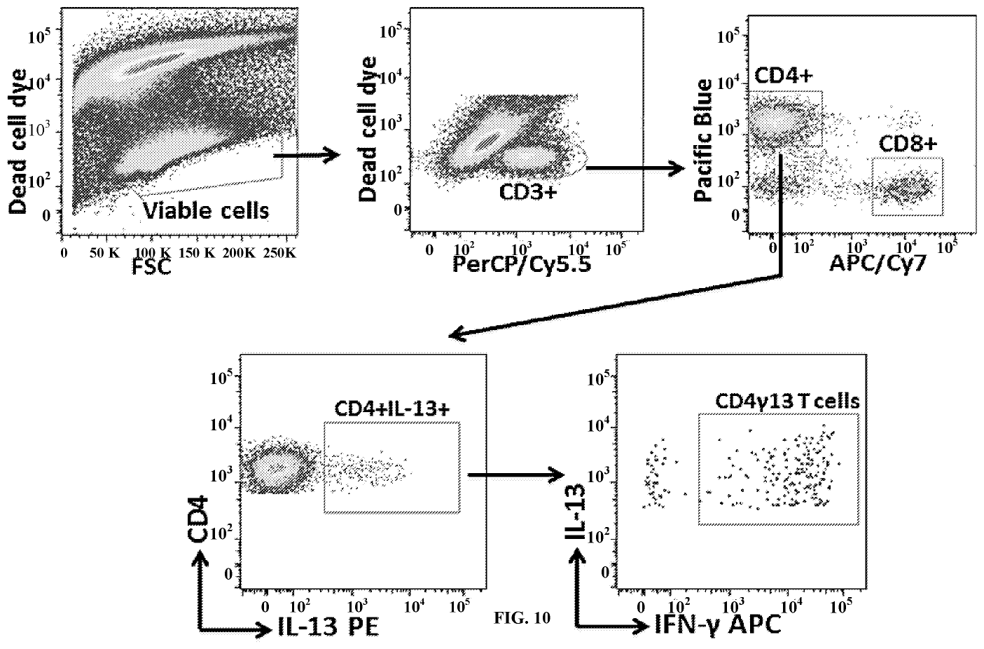

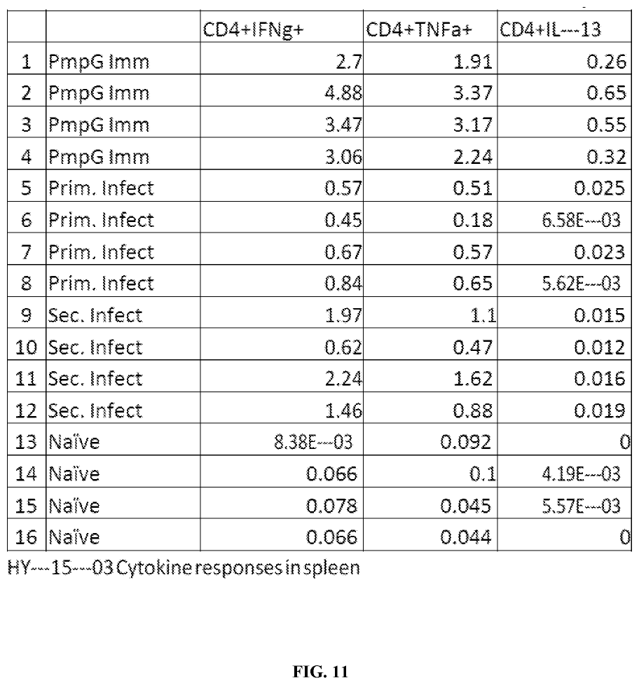

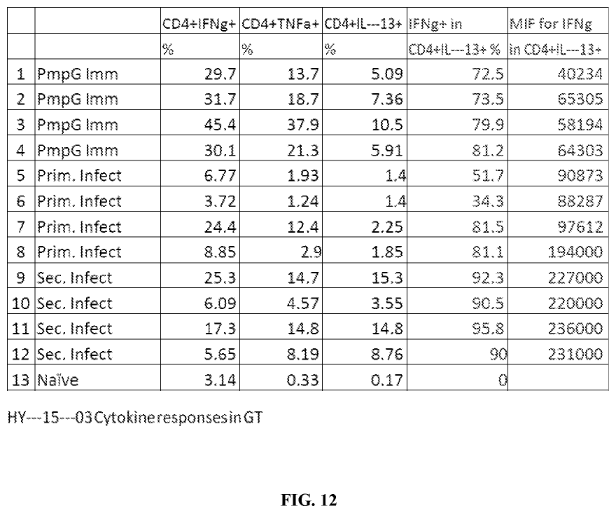

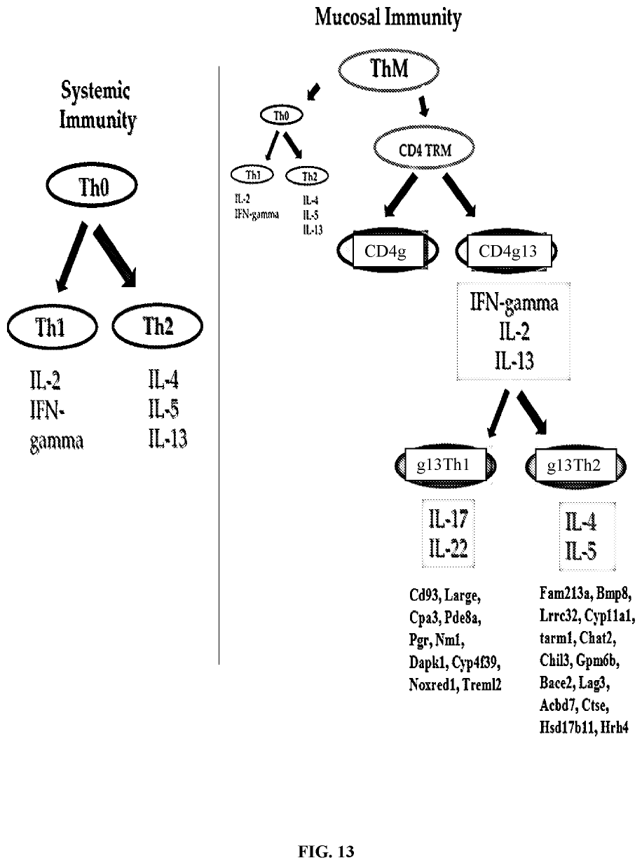

Next, CD4g13 T cell response to Chlamydia infection was investigated systemically (spleen) and locally (genital tract). CD4 T cell responses in the genital tract and spleen, quantified for IFN-g, TNFα, and IL-13, were determined for naive, PmpG-immunized, and immune mice (cleared prior infection) on day 6 post C. muridarum infection (FIG. 2C, gating strategy in FIG. 10; data for individual mice in FIG. 7). CD4g13 T cells were present but rare in the spleen. In the genital tract during primary infection 1-2% of CD4 T cells were IL-13+, increased to 5-10% with PmpG/DDA/TDB immunization, and were maximal during secondary infection 4-15%. In naïve mice, roughly half of the IL-13+CD4 T cells were IFN-g positive; 70-80% in PmpG immunized mice during a primary response; >90% dual cytokine positive in mice during a secondary response (FIG. 2D). The results demonstrated that CD4g13 T cells were a significant component of the local mucosal, but not the systemic, CD4 T cell response to primary genital tract infections, and their numbers were enhanced in the setting of pre-existing immunity due to prior infection or protective PmpG vaccination.

### Example 3: CD4g13 T Cell Clones

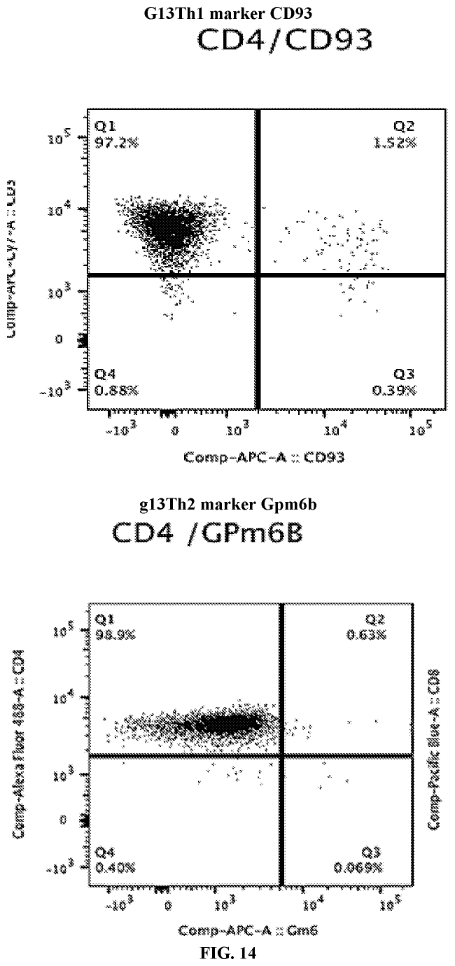

Having established that CD4g13 T cells were a physiologic component of the host response to Chlamydia genital tract infections, CD4 T cell clones were generated using immune B cell antigen presentation to assemble a panel of immune-B-cell-APC-derived multifunctional Th1 and CD4g13 T cell clones for comparison to each other, and to the existing CD4 T cell clones. Using conditions mimicking the polyclonal derivation, modified to incorporate TGFβ1, and using both uvMoPn and soluble Chlamydia antigen, a panel of T cell clones was derived from immune mice. A working panel of six CD4 T cell clones was carried forward including: two immune-B-cell-APC-derived CD4g13 clones (sBT13-7 & sBT16-8), two immune-B-cell-APC-derived multifunctional Th1 clones that did not produce IL-13 (BT12-7 & sBT13-11), an immune-B-cell-APC-derived CD4 clone that lost IL-13 production over time (BT12-17), and multifunctional Th1 clone derived with unfractionated-splenocyte-APC that was previously described (4uvmo-3) (Jayarapu et al., 2009, Infect Immun 77:4469-4479). The CD4 T cell clones were activated with immobilized anti-CD3 then levels of IL-2, IFN-g, IL-13, IL-10, TNFa, IL-17, IL-22, IL-4, and IL-5 in culture supernatant determined by ELISA (FIG. 3A). Both CD4g13 clones expressed IL-2, IFN-g, IL-13, IL-10, TNFa. One clone, sBT13-7 expressed IL-17 and IL-22, a Th1-like phenotype while the other clone, SBT16-8, expressed IL-4 and IL-5, a Th2-like phenotype. These two novel CD4 populations are also detectable in healthy human PBMCs, albeit at very low frequency (FIG. 14). CD93 was used to identify the Th1-like subset and GPm6B was used to identify the Th2-like subset. Th2 cells have a TCR-independent pathway for IL-13 production based on prostaglandin D binding the CrTh2 receptor (Xue et al., 2005, J Immunol 175:6531-6536). As CD4g13 T cells have not been previously described, IL-13 production was tested for a calcineurin-dependency, and to see whether the known calcineurin-independent prostaglandin D-CrTh2 pathway could account for IL-13 production (FIG. 3B). As compared to IFN-g, IL-13 production was significantly less inhibited by CsA. Also, based on small molecule inhibitors, the residual IL-13 production in the presence of CsA was not due to the CrTh2 pathway.

Next, the ability of CD4 T cell clones' to recognize and terminate C. muridarum replication in epithelial cells was investigated (FIG. 3C; top/middle panels), and their relative cytolytic ability was compared to a Chlamydia-specific CD8 CTL clone (8uvmo-2) using redirected lysis (FIG. 3C; bottom panel). All the CD4 T cell clones recognized infected epithelial cells, and to varying degrees terminated C. muridarum replication in them; only the CD4g13 clone sBT16-8 fell below 50% inhibition. Two CD4 clones terminated C. muridarum without IFN-g pre-treatment; 4uvmo-3 was nearly IFN-g-independent (did not require epithelial cells be pretreated with IFN-g) consistent with investigations (Jayarapu et al., 2009, Infect Immun 77:4469-4479). All CD4 clones produced Chlamydia-specific IFN-g under the conditions of the replication termination assay; IL-13 was not detected under these conditions. No conclusions can be drawn about IFN-g production versus termination efficiency as termination likely reduces T cell activation as suggested by lower levels of IFN-g for all CD4 T cell clones when epithelial cells were pretreated with IFN-g (improved termination efficiency); the highest level of IFN-g was for sBT13-11 with untreated infected epithelial cells (8196 pg/ml), an experimental condition in which sBT13-11 did not significantly terminate replication. The CD4 clones were less cytolytic than a conventional CD8 CTL clone, but all had some ability to kill in a short-term assay.

### Example 4: Gene Expression Micro Array Analysis of CD4g13 T Cells

Having a panel of CD4g13 and multifunctional Th1 T cell clones offered the possibility of defining CD4g13 T cells at the molecular level using gene expression microarray analysis. The initial investigation was done using T cells in their “rested” state as that condition was more likely to reflect T cell differentiation biology, i.e. biomarkers that may be useful in peripheral blood and uninfected tissue. T cells at the end of the usual seven day culture cycle were purified by ficoll-hypaque and plated without antigenic stimulation for an additional 48 h in media containing recombinant IL-7. Two days later the wells were harvested and total RNA isolated; the experiment was repeated 4 times to minimize false discovery. The comparators were sBT13-7 and sBT16-8 (CD4g13), BT12-7 and sBT13-11 (multifunctional Th1 derived with B cell APC that do not produce IL-13), BT12-17 (multifunctional Th1 that initially made then lost IL-13 production), and 4uvmo-3 (multifunctional Th1 derived with unfractionated splenocyte APC). The value of BT12-17 was unclear; it either represented plasticity in the CD4g13 phenotype or a breakthrough dominant second clone from incomplete limiting dilution. At worst BT12-17 was a third multifunctional CD4 T cell that did not produce IL-13. sBT13-7 and sBT16-8 were derived with soluble Chlamydia antigen; the other clones with uvMoPn. The microarray comparisons were as follows: (sBT13-7 & sBT16-8) vs (BT12-7 and sBT13-11): CD4g13 vs multifunctional Th1 [all B cell-derived](sBT13-7 & sBT16-8) vs (BT12-17): possible unique insight into IL-13 biology (loss of function) (sBT13-7 & sBT16-8) vs (4uvmo-3): CD4g13 vs conventional multifunctional Th1 [splenocyte APC derived]. The criteria applied to identify genes of interest were a) a log 2 fluorescence signal>5.0 (mRNA signal above background), b) a statistically significant (p value<0.01) 3-fold (up or down) difference between the two CD4g13 clones in aggregate versus non-CD4g13 clones in all three comparisons, and c) the log 2 fluorescence signal for both CD4g13 clones had to be greater than the individual log 2 signals for all the other T cell clones in the array (eliminates genes skewed by very high or low expression by one of the CD4g13 T cell clones). Analysis of the microarray data showed that the CD4g13 T cell clones had more genes in common with each other than the other clones (FIG. 4). Genes with significantly enhanced mRNA signal in CD4g13 T cells are shown in Table 1 below; those with significantly reduced mRNA signal are shown in Table 2 below. All gene mRNA differences in Table 1 and Table 2 were statistically significant with the highest false discovery rates (FDR) being 7×10⁴ (Trib2) for the up genes and 1×10\hat{ }(S1pr1) for the down genes.

TABLE 1

| | | Fold- | | | | | |
|---|---|---|---|---|---|---|---|
| | p-value | Change | | Fold- | | Fold- | |
| | (13-7 & | (13-7 & | p-value | Change | p-value | Change | |
| | 16-18 vs | 16-18 vs | (13-7 & | (13-7 & | (13-7 & | (13-7 & | |
| Gene | 12-7 & | 12-7 & | 16-18 vs | 16-18 vs | 16-18 vs | 16-18 vs | |
| Symbol | 13-11) | 13-11) | 12-17) | 12-17) | 4uvmo-3) | 3-10) | gene title |
| Bace2 | 2.29E−12 | 16.89 | 6.73E−11 | 14.76 | 1.59E−10 | 12.78 | beta-site APP-cleaving enzyme 2-transmembrane |
| | | | | | | | protease |
| Eomes | 1.62E−12 | 14.73 | 7.94E−14 | 45.01 | 6.07E−14 | 47.92 | eomesodermin homolog (Xenopus laevis)-prevents |
| | | | | | | | CD4−> Treg; makes CD8 less cytolytic |
| Trat1 | 5.03E−08 | 10.11 | 4.80E−07 | 10.13 | 1.31E−07 | 12.74 | T cell receptor associated transmembrane |
| | | | | | | | adaptor 1-adjust TCR signaling |
| Acvr2a | 6.70E−11 | 7.72 | 3.46E−10 | 8.71 | 1.31E−08 | 5.49 | activin receptor IIA- T cell differentiation |
| | | | | | | | (Th17) IL-6 + TGFbeta |
| Nrn1 | 7.39E−11 | 6.35 | 3.69E−09 | 5.41 | 3.33E−09 | 5.47 | neuritin 1-transcript found in CD8 TIL(tumor |
| | | | | | | | infiltrating lymphocytes) and Treg |
| Ctse | 1.41E−10 | 6.29 | 4.52E−08 | 4.37 | 3.59E−07 | 3.57 | cathepsin E-releases TRAIL in Tregs |
| Cpa3 | 4.97E−09 | 6.26 | 7.83E−10 | 11.46 | 6.68E−08 | 6.09 | carboxypeptidase A3, mast cell-released |
| | | | | | | | granule protease |
| Lrrc32 | 4.70E−07 | 6.19 | 1.77E−06 | 6.96 | 4.28E−07 | 8.67 | leucine rich repeat containing 32-binds latent |
| | | | | | | | TGFb complex |
| Wls | 2.40E−11 | 5.96 | 4.71E−10 | 5.69 | 8.16E−14 | 19.78 | wntless homolog (Drosophila)-wnt ligand |
| | | | | | | | secretion vehicle; IL-13, IL-4; |
| 2900026A02Rlk | 9.62E−11 | 5.40 | 1.78E−10 | 6.73 | 2.42E−09 | 5.00 | RIKEN cDNA 2900026A02 gene-intracellular |
| | | | | | | | protein unknown function |
| Gpx8 | 2.26E−11 | 5.23 | 3.66E−13 | 12.36 | 3.52E−13 | 12.43 | glutathione peroxidase 8 (putative)-er |
| | | | | | | | protein-disulfide bonds-regulated by EPas1 |
| Ccr8 | 6.27E−10 | 5.17 | 9.44E−11 | 8.88 | 7.88E−15 | 49.24 | chemokine (C-C motif) receptor 8 |
| Lrrc16a | 1.61E−10 | 4.73 | 3.68E−09 | 4.44 | 1.10E−08 | 4.00 | luecine rich repeat containing 16A-uncaps |
| | | | | | | | actin-slows migration |
| Rgs10 | 6.69E−12 | 4.68 | 1.11E−14 | 14.67 | 3.61E−15 | 17.72 | regulator of G-protein signalling10-knockout |
| | | | | | | | with reduced CD4 with aging |
| Oit3 | 3.98E−10 | 4.52 | 3.55E−10 | 5.98 | 4.36E−07 | 3.00 | oncoprotein induced transcript 3-secreted |
| | | | | | | | protein |
| Fhl2 | 8.86E−09 | 4.05 | 1.96E−09 | 6.16 | 3.82E−10 | 7.56 | four and a half LIM domains 2-knockout mouse |
| | | | | | | | has decreased IL13 response |
| Epas1 | 1.40E−07 | 3.95 | 8.68E−11 | 14.14 | 4.44E−08 | 5.77 | endothelial PAS domain protein 1-transcription |
| | | | | | | | factor assoc with cytokines |
| Trib2 | 2.23E−06 | 3.56 | 4.73E−05 | 3.18 | 3.08E−05 | 3.33 | tribbles homolog 2 (Drosophila)-cell survival |
| Qpct | 1.37E−06 | 3.45 | 4.81E−09 | 8.81 | 3.55E−09 | 9.21 | glutaminyl-peptide cyclotransferase (glutaminyl |
| | | | | | | | cyclase)-n terminal protein modification |
| Bcl2l11 | 2.28E−10 | 3.32 | 1.60E−09 | 3.47 | 1.72E−15 | 18.58 | BCL2-like 11 (apoptosis facilitator)-Cytokine |
| | | | | | | | withdrawl upregulates apoptosis |
| Afap1l1 | 2.33E−06 | 3.31 | 2.40E−05 | 3.20 | 2.21E−05 | 3.23 | actin filament associated protein 1-like |
| | | | | | | | 1-rounded cell shape |
| Gnb4 | 1.30E−09 | 3.08 | 2.41E−12 | 7.21 | 1.54E−13 | 10.36 | guanine nucleotide binding protein (G protein), |
| | | | | | | | beta 4-LFA-1 activation |

TABLE 2

| | | Fold- | | | | | |
|---|---|---|---|---|---|---|---|
| | p-value | Change | | Fold- | | Fold- | |
| | (13-7 & | (13-7 & | p-value | Change | p-value | Change | |
| | 16-8) vs | 16-18) vs | (13-7 & | (13-7 & | (13-7 & | (13-7 & | |
| Gene | (12-7 & | (12-7 & | 16-18) vs | 16-18) vs | 16-18) vs | 16-18) vs | |
| Symbol | 13-11) | 13-11) | (12-17) | (12-17) | (4uvmo-4) | (4uvmo-3) | gene title |
| Clec5a | 4.11E−07 | −5.47 | 2.43E−09 | −17.37 | 2.19E−04 | −3.25 | C-type lectin domain family 5, member |
| | | | | | | | a-activated by viruses |
| Fbxo27 | 1.47E−10 | −4.95 | 4.55E−10 | −5.75 | 1.72E−12 | −12.04 | F-box protein 27-targeting for |
| | | | | | | | ubiquitin ligase (function unknown) |
| Ggt5 | 5.93E−10 | −3.80 | 1.36E−11 | −7.37 | 1.17E−08 | −3.63 | gamma-glutamyltransferase 5-leukotriene |
| | | | | | | | D4 production |
| Lima1 | 2.34E−12 | −3.79 | 2.39E−14 | −7.97 | 5.58E−14 | −7.19 | LIM domain and actin binding 1-actin |
| | | | | | | | depolimerization |
| Ptgdr2 | 6.04E−09 | −3.36 | 2.60E−08 | −3.64 | 5.91E−13 | −13.03 | prostaglandin D2 receptor 2-CrTh2 TCR |
| | | | | | | | independent IL-13 production |
| S1pr1 | 2.57E−06 | −3.31 | 2.33E−12 | −36.54 | 9.88E−12 | −26.83 | sphingosine-1-phosphate receptor |
| | | | | | | | 1-recruits T cells into circulation |
| Tln2 | 2.63E−10 | −3.15 | 2.89E−11 | −4.72 | 1.97E−12 | −6.26 | talin 2-assembly of actin filaments |

The cytokine data for CD4g13 T cell clones (see FIG. 3A) showed unusual combinations of Th1/Th2/Th17/Th22 cytokines so CD4g13 T cell clone differentiation markers/transcription factors were parsed out from the microarray (See Table 3 below).

TABLE 3
Lineage and Differentiation markers

| | | CD4γ13 T cell clones | | | |
|---|---|---|---|---|---|
| | | mRNA signal | | | |
| | | (arbitrary units) | | | |
| | | Mean (13-7) | Mean (16-18) | Score* | gene title |
| Lineage | Cd4 T cells | 7651 | 9484 | ++++ | CD4 antigen |
| | Cd8 T cells | 51 | 56 | 0 | CD8 antigen, beta chain 1 |
| | NK/NKT cells | 82 | 27 | 0 | killer cell lectin-like receptor subfamily B mem 1C |
| | B cells | 40 | 41 | 0 | CD79A antigen (immunoglobulin-associated alpha) |
| Cytokine | Th1 | 523 | 402 | + | T-box 21 (T-bet) |
| polarization | Th2 | 1677 | 4184 | ++/+++ | GATA binding protein 3 (Gata3) |
| | Th17 | 62 | 45 | 0 | RAR-related orphan receptor gamma (RORγT) |
| | Th22 | 283 | 96 | +/0 | aryl-hydrocarbon receptor (Ahr) |
| | ? | 298 | 855 | + | endothelial PAS domain protein 1 (Epas1) |
| | ? | 1456 | 3741 | ++/+++ | eomesodermin (Eomes) |
| Trm | Klrg1 | 64 | 57 | 0 | killer cell lectin-like receptor subfamily G, mem 1 |
| | Klf2 | 113 | 88 | 0 | Kruppel-like factor 2 (lung) |
| | Hnf1a | 90 | 102 | 0 | HNF1 homeobox A |
| | S1pr1 | 90 | 61 | 0 | sphingosine-1-phosphate receptor 1 |
| | Ccr7 | 252 | 268 | + | chemokine (C-C motif) receptor 7 |
| | Zfp683 | 52 | 71 | 0 | zinc finger protein 683 (Hobit) |
| | Prdm1 | 1121 | 772 | ++/+ | PR domain containing 1, (Blimp-1) |
| | Rgs1 | 2318 | 2972 | ++/+++ | regulator of G-protein signaling 1 |
| | Rgs2 | 1032 | 2238 | ++ | regulator of G-protein signaling 2 |
| | Cd69 | 3526 | 3169 | +++ | CD69 antigen |
| | Cd44 | 5222 | 4794 | +++ | CD44 antigen |
| | Itgal | 5688 | 6527 | +++ | integrin alpha L |
| | Itga4 | 1605 | 214 | ++/+ | integrin alpha 4 |
| | ltgb7 | 3184 | 2423 | +++ | integrin beta 7 |
| | Itga1 | 1021 | 701 | ++/+ | integrin alpha 1 |
| | Itgae | 265 | 784 | + | integrin alpha E, epithelial-associated |
| Treg | Cd27 | 25 | 23 | 0 | CD27 antigen |
| | tl2ra | 1070 | 1285 | ++ | interleukin 2 receptor, alpha chain |
| | Foxp3 | 108 | 136 | 0 | forkhead box P3 |
| | Ctla4 | 2127 | 1519 | ++ | cytotoxic T-lymphocyte-associated protein 4 |
| Th22 | Ahr | 283 | 96 | +/0 | aryl-hydrocarbon receptor |
| | Foxo4 | 179 | 156 | +/+ | forkhead box O4 |
| | Bnc2 | 43 | 39 | 0 | basonuclin 2 |

*<150 au (~3x the CD8b signal) = 0, 150-999 = +, 1000-2499 = ++, 2500-7499 = +++, 7500-10,000 = ++++

Additionally, a western blotting was performed for Tbet (Th1), Gata3 (Th2), Eomes, and Fhl2 on the two CD4g13 T cell clones (sBT13-7 and sBT16-8) and three IL-13 negative controls (4uvmo-3, BT12-7, BT12-17) on day 5 of their usual 7 day culture cycle (peak T cell numbers in well). Differentiation markers/transcription factors in the microarray with mRNA signals that were negligible, RORyT (Th17), or low and mismatched between the two CD4g13 clones, Ahr (Th22), were not included in western blot analysis; blotting with commercial antibodies for Epasl generated low quality blots and therefore not included in FIG. 5. Transcriptionally the CD4g13 T cell clones looked like Trm (Klrg1^{neg}, Klrg2^{neg}, Hnfla^{neg}, S1pr1^{neg}, Ccr7^{neg}; Hobit^{neg}, Blimp-1^{pos}, Rgs1^{pos}, Rgs2^{pos}, CD69^{pos}, CD44^{pos}). Chlamydia-specific multifunctional Th1 derived from mice that self-cleared a genital tract infection universally expressed Gata3 and Eomes. Interestingly, Tbet expression, as detectable at the level of western blotting, did not appear to be required for IFN-g production though its relative absence may be required for IL-4 production (e.g. sBT16-8), and Gata3 in Chlamydia-specific multifunctional Th1 did not denote a conventionally defined Th2 phenotype or IL-4 production. Fhl2 appeared to be the transcription factor that qualitatively, and perhaps quantitatively, denoted a CD4g13 T cell's ability to uniquely produce IL-13.

### Example 5: Adoptive Transfer of Multifunction Th1 Versus CD4g13

To determine whether CD4g13 T cells were capable of protecting or causing genital tract pathology during C. muridarum infections, these cells were adoptively transferred into naive C57BL/6 mice and challenged the next day with genital tract infections. For comparison, 4uvmo-3, which is the conventional multifunctional Th1 comparator that was previously predicted to be protective based on Plac8 positivity was adoptively transferred. Early and relatively IFN-g independent recognition of infected epithelial cells and efficient termination of Chlamydia replication was assessed (Johnson et al., 2012, J Immunol 188:1896-1904). The initial experiment, and a staggered/stacked replicate second experiment, were focused on 4uvmo-3 (splenic-APC-derived multifunctional Th1) versus sBT16-8 (CD4g13 with highest IL-13 production), piloting smaller numbers of mice with sBT13-7 (other CD4g13 clone) and BT12-17 (B cell-derived multifunctional Th1 without IL-13). When the first experiment reached 8 weeks and was scored for pathology it was clear that sBT13-7 was likely the most protective T cell clone (zero pathology in three mice; control incidence is >60%). A third cohort of control and sBT13-7 mice was initiated to complete the data set. Mice were monitored for bacterial shedding through day 30 (FIG. 6A and FIG. 6B) and killed on day 56 to score immunopathology (FIG. 6C; dissected genital tracts in FIG. 6D). The multifunctional Th1 clone 4uvmo-3 was partially protective, reducing the frequency of hydrosalpinx from ˜60% of oviducts to ˜20%. sBT13-7, a CD4g13 T cell clone, dramatically protected mice from C. muridarum immunopathology, preventing damage to uteri in 8 of 9 mice and preventing hydrosalpinx in 9 of 9 mice. sBT16-8, the other CD4g13 T cell clone, was neither protective or pathologic. Interestingly the protection afforded to the murine genital tract by sBT13-7 and 4uvmo-3 was largely limited to oviducts and did not correlate with rapidity of bacterial clearance.

### Example 6: CD4g13 Cells Molecular Fingerprints

In the context of an emerging new understanding of mucosal host defense based on local adaptive immunity mediated by tissue resident memory T cells (Trm), it was discovered herein that Chlamydia genital tract pathogenesis is a Trm rather than a cytokine polarization Th1/Th2/Th17 framework. It was also shown herein that the Chlamydia-specific CD4 T cell response includes a population of CD4 T cells that produce IFN-g and IL-13 and that the Chlamydia memory lymphocyte clusters include immune plasma B cells as antigen presenting cells (APC). Specifically, the present invention relates to the discovery and characterization of CD4g13 T cells.

The novel CD4g13 cells of this invention ignore the mutual exclusivity rules of T cell differentiation defined by studies of the systemic immune compartment which is driven by IFN-gamma or IL-4 and Th0 differentiates into either Th1 or Th2 respectively. In the case of the mucosal immunity, the CD4g13 cells are driven by a cytokine milieu that includes TGFβ to polarize to IFN-gamma or IFN-gamma and IL-13 (Th2 cytokine). Additionally, the CD4g13 T cell subset further divide into two groups (phenotypes), either g13Th2 or g13Th1 based on additional production of either IL-4/5 or IL-17/22 respectively.

Characterization of the molecular fingerprints for both g13Th2 and g13Th1 cells (listed below herein in Tables 4 and 5 respectively) was accomplished using data from a gene expression microarray. The molecular footprint includes for instance cell surface proteins (targets for antibody-based therapeutic biologics) and enzymes (small molecule inhibitors).

TABLE 4
List of g13Th2 biomarkers:
Fam213a, Bmp8, Lrrc32, Cyp11a1, tarm1, Chat2, Chil3, Gpm6b, Bace2, Lag3,
Acbd7, Ctse, Hsd17b11 and Hrh4.

| | | | | Fold- | |
|---|---|---|---|---|---|
| | | | FDR (16-18 | Change | |
| | | Fold- | vs. 5 CD4 | (16-18 vs. | |
| CD4g13-4/5 | FDR | Change | T cell | 5 CD4 T | |
| Gene | (16-8 | (16-8 vs | clone | cell clone | |
| Symbol | vs 13-7) | 13-7) | panel) | panel) | gene |
| Fam213a | 2.E−14 | 25.09 | 1.E−15 | 18.29 | family with sequence similarity 213, member A* |
| Bmp7 | 5.E−08 | 15.06 | 1.E−09 | 13.76 | bone morphogenetic protein 7* |
| Lrrc32 | 2.E−05 | 14.75 | 5.E−08 | 22.78 | leucine rich repeat containing 32** |
| Cyp11a1 | 7.E−07 | 11.86 | 1.E−08 | 11.52 | cytochrome P450, family 11, subfamily a, polypeptide 1 ‡ |
| Tarm1 | 7.E−09 | 11.30 | 4.E−11 | 13.19 | T cell-interacting, activating receptor on myeloid cells 1 † |
| Chst2 | 1.E−07 | 7.77 | 2.E−10 | 11.70 | carbohydrate sulfotransferase 2- (rheumatoid arthritis biomarker) ‡ |
| Chil3 | 1.E−03 | 6.88 | 2.E−05 | 8.20 | chitinase-like 3* |
| Gpm6b | 6.E−05 | 4.71 | 4.E−07 | 5.57 | glycoprotein m6b † |
| Bace2 | 1.E−04 | 4.40 | 2.E−10 | 21.26 | beta-site APP-cleaving enzyme 2 ‡ |
| Lag3 | 9.E−03 | 4.30 | 8.E−05 | 6.00 | lymphocyte-activation gene 3 † |
| Acbd7 | 3.E−07 | 3.24 | 1.E−08 | 3.00 | acyl-Coenzyme A binding domain containing 7 ‡ |
| Ctse | 4.E−03 | 3.19 | 6.E−07 | 6.85 | cathepsin E ‡ |
| Hsd17b11 | 4.E−04 | 3.14 | 1.E−08 | 7.12 | hydroxysteroid (17-beta) dehydrogenase 11 ‡ |
| Hrh4 | 2.E−04 | 3.14 | 3.E−07 | 4.48 | histamine receptor H4** |

*secreted protein,
**receptor,
† cell surface,
‡ enzyme activity

TABLE 5
List of g13Th1 biomarkers:
Cd93, Large, Cpa3, Pde8a, Pgr, Nm1, Dapk1, Cyp4f39, Noxred1 and Treml2.

| | | | | Fold- | |
|---|---|---|---|---|---|
| | | | FDR (13-7 | Change | |
| | | Fold- | vs. 5 CD4 | (13-7 vs. | |
| | FDR | Change | T cell | 5 CD4 T | |
| CD4gl3-17/22 | (13-7 | (13-7 vs | clone | cell clone | |
| Gene Symbol | vs 16-8) | 16-8) | panel) | panel) | gene |
| Cd93 | 9.E−10 | 27.60 | 9.E−11 | 21.96 | CD93 antigen † |
| Large | 1.E−09 | 20.77 | 4.E−10 | 12.57 | like-glycosyltransferase ‡ |
| Cpa3 | 6.E−08 | 18.81 | 4.E−10 | 28.28 | carboxypeptidase A3, mast cell ‡ |
| Pde8a | 7.E−08 | 15.11 | 2.E−08 | 10.32 | phosphodiesterase 8A ‡ |
| Pgr | 2.E−07 | 10.86 | 5.E−09 | 11.48 | progesterone receptor** |
| Nrn1 | 3.E−07 | 8.77 | 5.E−10 | 15.58 | neuritin 1 † |
| Dapk1 | 4.E−06 | 7.55 | 1.E−06 | 5.23 | death associated protein kinase 1 ‡ |
| Cyp4f39 | 1.E−03 | 4.53 | 8.E−04 | 3.41 | cytochrome P450, family 4, subfamily f, polypeptide 39 ‡ |
| Noxred1 | 3.E−05 | 3.57 | 6.E−06 | 3.06 | NADP+ dependent oxidoreductase domain containing 1 ‡ |
| Treml2 | 5.E−02 | 3.18 | 2.E−03 | 4.44 | triggering receptor expressed on myeloid cells-like 2 † |

*secreted protein,
**receptor,
† cell surface,
‡ enzyme activity

### Example 6: Overview

B cells, CD4 T cells, and IL-13 were investigated during Chlamydia infections of the genital tract in the context of tissue resident mucosal immunity. The incentive for this investigation was the paradoxical data regarding Th2 cells and IL-13 in Chlamydia host defense. On one hand Th2 responses to Chlamydia infections have been associated with ineffectual or worsened pathological outcomes in mouse models (Gondek et al., 2009, J Immunol 183:1313-1319; Hawkins et al., 2002, Infect Immun 70:5132-5139), including a study showing that IL-13 knockout mice cleared infections more rapidly with less pathology than wild type mice (Asquith et al., 2011, PLoS Pathog 7:e1001339). On the other hand, human clinical investigation showed that a PBMC IL-13 response to EB prospectively identified individuals resistant to reinfection with C. trachomatis (Cohen et al., 2005, J Infect Dis 192:591-599), supporting a protective role for presumably T cells polarized to produce IL-13. The data presented here revealed that these disparate results are biologically compatible, and called into question the utility of the Th1/2/17 cytokine polarization framework for understanding genital tract immunity during Chlamydia infections. Mice deficient in B cells clear primary C. muridarum infections in the usual time frame with the caveat that they develop a transient peritonitis and early dissemination (Li et al, 2013, PLoS Pathog 9:e1003707); mice deficient in B cells remain susceptible to reinfection with a clearance time only slightly faster than that of naive mice during primary infections (Su et al., 1997, Infect Immun 65:1993-1999). Published immuno-histochemical analysis of lymphoid aggregates in human and mouse genital tract and conjunctival tissues in the setting of current or prior Chlamydia infections documents abundant B cells in Chlamydia-specific memory lymphocyte clusters (c-MLC) (reviewed by Johnson et al., 2016 Infect Immun 84:868-873). The combination of B cell knockout mouse susceptibility to reinfection and the abundance of B cells in c-MLC suggests that B cells play an important role in protective secondary immune responses (Li et al., 2015, Immunol Lett 164:88-93). In the original mouse work by Morrison and Morrison based on B220 immunohistochemical staining, B cells were present during the first two weeks of infection but disappeared in subsequent weeks. B220 is down-regulated as activated B lymphocytes transition to immune plasma B cells (Dustin et al., 1995, J Immunol 154:4936-4949). The status of B lymphocytes and plasma B cells was analyzed herein over the time course of infection by doing flow cytometry on single cell suspensions generated from uteri and oviducts. Pan-B cell marker CD79a was used to identify B cells in toto, and B220 to characterize them as B lymphocytes (B220 hi) or plasma B cells (B220 lo). Plasma B cells were found to be nearly absent in naive genital tract tissue, became detectable during primary infection, and markedly expanded during infections in mice with pre-exiting immunity generated either by PmpG/DDA/TDB vaccination or prior infection. The kinetics of plasma B cell expansion mirrored the time course of demonstrable T cell immunity to C. muridarum (Su et al., 1999, J Infect Dis 180:1252-1258), and were compatible with plasma B cells playing a role in Chlamydia-specific MLC (c-MLC) and protective immunity. These results presented herein suggest that a novel T cell recovery/expansion protocol based on immune B cell APC is physiologically relevant. Immune B cell presentation of Chlamydia antigens to T cells from mice that previously cleared genital tract infections preferentially expanded CD4 T cells over CD8 T cells, including a subset polarized to produce IL-13 and IFN-g. Analysis of CD4 T cells in the mouse genital tract and spleen in the naive state, vaccinated state, and post clearance of a primary C. muridarum infection, showed that CD4g13 T cells were localized to the genital tract in physiologically relevant levels; up to 15% of Chlamydia-specific T cells, a 500-fold enrichment of CD4g13 T cells in the genital tract tissue versus the spleen. Importantly, immunization with the protective PmpG in DDA/TDB vaccine at the base of the tail enhanced the presence of CD4g13 T cells in the genital tract. Using the immune B cell APC protocol, a panel of CD4 T cell clones were generated from the spleens of immune mice (prior genital tract infection) that included two CD4g13 T cells, thereby providing an opportunity to define the CD4g13 T cell subset at the molecular level using gene expression microarray analysis. The microarray data suggested a CD4g13 memory T cell subset with Trm-like differentiation (Klrg1^{neg}, K//2^{neg}, Hnfla^{neg} S1pr1^{neg}, Ccr7^{neg}; Hobit^{neg} Rgs1^{pos}, Rgs2^{pos}, CD69^{pos}, CD44^{pos}) (Mackay et al., 2017, Trends Immunol 38:94-103). It is proposed herein that the CD4g13 T cell clones are progeny of the contraction of the CD4 T cell response during the primary genital tract infection. Trm trafficked to the spleen to serve as a Trm repository reflecting events that occurred in the genital tract. Within the microarray CD4g13 T cell clones were more like each other than the other T cells in the panel, but were not homogenous. At the cytokine level sBT13-7 and sBT16-8 shared at least 5 cytokines (IL-2, IFN-g, IL-13, IL-10, TNFa), with sBT13-7 adding IL-17 and IL-22, and sBT16-8 adding IL-4 and IL-5. Unlike IL-2/IL-4/IFN-g, T cell production of IL-13 is partially resistant to cyclosporine A (CsA). In human peripheral blood CD4 T cells CsA-resistant IL-13 production occurs only at low concentrations, <30 nM, through a MEK-dependent pathway (Pahl et al., 2002, Br J Pharmacol 135:1915-1926). The CD4g13 T cells reported here continued to preferentially produce IL-13 versus IFN-g in experiments using 500 nM CsA, >10 times the concentration that completely blocked IL-13 in human peripheral blood CD4 T cells. It was also tested whether the Th2 calcineurin-independent (CsA-resistant) prostaglandin D—CrTh2 pathway (Xue et al., 2005, J Immunol 175:6531-6536) was responsible for CsA-resistant IL-13 production in the CD4g13 T cell clones. The results showed that this was not the case, and the microarray analysis showed diminished CrTh2 mRNA transcripts in CD4g13 compared to multifunctional Th1 clones (Table 2). These data suggest that CD4g13 T cells have a TCR signaling pathway that regulates IL-13 production independent of calcineurin/NFAT activation. The differentiation/cytokine polarization of Chlamydia-specific IFN-g producing CD4 T cells generated during clearance of Chlamydia genital tract infections appears to be based on the transcription factors Gata3 and Eomes, and usually but not always Tbet. A previous study showed few Gata3+ CD4 T cells in spleen and lymph nodes during C. muridarum infection without investigation of genital tract tissue (Li et al, 2013, PLoS Pathog 9:e1003707). A human study showed CD4 Gata3+ T cells in presumed memory lymphocyte clusters in endometrial biopsies from Ct infected women but not uninfected controls (Vicetti et al., 2013, PLoS One 8:e58565). In isolation two CD4g13 T cell clones and four multifunctional CD4 T cell clone controls are not sufficient to draw definitive conclusions about T cell biology. The present results are consistent with the existing paradigm that Tbet (Th1) must be downregulated for a CD4g13 T cell clone to produce IL-4 (Th2); e.g. sBT16-8. More importantly, it was shown herein that Fhl2 was CD4g13-associated at the transcript level (microarray) and protein level (western blot). Because Fhl2 knockout mice have a deficit in IL-13 production (Kurakula et al., 2015. Allergy 70:1531-1544), it is reasonable to postulate that Fhl2 is the transcription factor that denotes a CD4g13 T cells' ability to produce IL-13, and Fhl2 identifies an ideal candidate pathway for calcineurin-independent IL-13 production. Though not conventionally quantifiable, immune B cell antigen presentation and exogenous TGFP1 were shown herein to be necessary to recover CD4g13 T cells and to maintain their IL-13 production ex vivo (FIG. 9). Absent TGFβ1, IL-13 production seen in early passages of polyclonal T cells faded with serial passage suggesting that in vivo CD4g13 memory T cells reside in a micro environment with active TGFβ1. Epithelial cells make latent TGFβ constitutively, and TGFβ in the latent form is abundant in mucosal tissues. The CD4g13 T cell molecular fingerprint (microarray) includes genes relevant to TGFβ biology including Lrrc16a, binding receptor for the latent TGFβ complex, Bace2 and Cpa3 proteases that potentially process latent TGFβ into active form for use in an autocrine/paracrine fashion, and Avr2a, receptor for activins that share some TGFβ signaling pathways. These genes may provide new insights into the unique biology of immune B cells and memory lymphocyte clusters within the genital tract. One of the most important findings in the present investigation is that CD4g13 T cell clone sBT13-7 completely protected oviducts from pathology in adoptive transfer. C. muridarum challenge experiments, as did to a lesser extent the multifunctional Th1 clone 4uvmo-3. Those results link multifunctional Th1 and CD4g13 T cells to protective immunity, with the interesting difference being that CD4g13 sBT13-7 had a Trm mRNA fingerprint (Klfn^{neg}, S1pr1^{neg}) compared to Th1 4uvmo-3 (Kf2^{pos}, S1PR1^{pos}). The CD4g13 phenotype in-and-of itself was not sufficient for genital tract protection as the CD4g13 sBT16-8 clone was not protective; sBT16-8 was also relatively ineffective terminating Chlamydia replication in epithelial cells in vitro. The mechanism of oviduct protection for 4uvmo-3 and sBT13-7 was not accelerated bacterial clearance as measured in the lower genital tract; 4uvmo-3 mice cleared at same rate as naive mice, while sBT13-7 mice had greater bacterial shedding at late time points, a result that may eventually provide insights into why IL-13 knockout mice clear infections faster than wild type mice (Asquith et al., 2011, PLoS Pathog 7:e1001339). At the level of individual T cell clone-mediated immunity, in wild type mice with normal immune systems, this study showed that protection from pathology and bacterial shedding can be dissociated. This dissociation has been previously demonstrated in mouse models with plasmid-deficient C. muridarum (O'Connell et al. 2007, J Immunol 179:4027-4034) and knockouts of TLR2 (Darville et al., 2003, J Immunol 171:6187-6197) and IL-1b (Prantner et al., 2009, Infect Immun 77:5334-5346). sBT13-7 protection from immunopathology implies a regulatory mechanism that influenced the naive wild type adaptive immune response, perhaps moderating the primary CD8 T cell response associated with immunopathology (Igietseme et al., 2009, J Infect Dis 200:926-934; Murthy et al., 2011, Infect Immun 79:2928-2935; Vlcek et al., 2016, Immunol Cell Biol 94:208-212). Both CD4g13 T cell clones have enhanced mRNA transcript levels for several Treg-associated genes including Nrnl, Ctse, and Lrrc32, however they are not Tregs as they produced IL-2 upon activation and neither had an mRNA signal for Foxp3. Thus it seems unlikely that Treg- or iTreg mechanisms fully account for protection from pathology mediated by sBT13-7. sBT13-7 somehow reduced neutrophil recruitment and/or promoted a more beneficial healing response within the TNFα-IL13-TGFβ axis (Fichtner-Feigl et al., 2006, Nat Med 12:99-106; Fichtner-Feigl et al., 2008, Gastroenterology 135:2003-2013, 2013 e2001-2007). Recent work by Li et al demonstrated that CCR7 homing contributes to the paucity T lymphocytes in the naive murine female genital tract, presumably by lymph node sequestration. Naive CCR7 knockout mice have an aberrant immune-architecture with many more T cells localized in genital tract tissue and cleared C. muridarum more rapidly and with less acute inflammation than did wild type mice (Li Let al., 2017, J Immunol doi:10.4049/jimmuno1.1601314). In the present study, adoptive transfer of the CD4g13 T cell clone sBT13-7 into wild type mice, with their usual paucity of immune architecture and T cells, prevented oviduct immunopathology. The CD4g13 T cell clones had enhanced CCR8 (5-12 fold higher; see Table 1) with reduced CCR7 mRNA transcripts (Table 3 and GEO data) compared to the four non-CD4g13 clones. Therefore, it is possible that CCR8, associated with skin homing (McCully et al., Blood 120:4591-4598), plays a role in c-MLC. While the mechanism of sBT13-7 protection remains to be determined, during primary genital tract infections in wild type mice it is reasonable to postulate that “how” is at least or more important than “how fast” Chlamydia is cleared, with implications for assessment of future. It is unlikely that IL-13 directly participates in the physical termination of Chlamydia replicating in reproductive tract epithelium as it was shown that IL-13 modestly enhances C. muridarum replication in an upper reproductive tract epithelial cell line (Johnson et al., 2014 Immunology 142:248-257), and that IL-13 knockout mice display accelerated bacterial clearance from the genital tract (Asquith et al., 2011, PLoS Pathog 7:e1001339). Instead, IL-13 was postulated as a biomarker for a CD4 Trm subset capable of preventing immunopathology during clearance of genital tract infections, and small numbers of CD4g13 T cells in circulation were proposed to be the source of EB stimulated PBMC IL-13 production that predicted resistance to Ct infection in the Cohen et al study of Kenyan female sex workers. Further studies of CD4g13 T cells in the mouse model provide the tools necessary to test those hypotheses in humans. Recently it has been proposed that protective/healing Th2 immunity explains how the majority of humans clear Chlamydia genital tract infections without fertility-limiting immunopathology based on Gata3-centered data (Vicetti et al., 2013, PLoS One 8:e58565; Vicetti et al., 2012, Med Hypotheses 79:713-716; Vicetti et al. 2017, Infect Immun 85; Vicetti et al., 2017, Proc Natl Acad Sci USA doi:10.1073); that Th2 conclusion is consistent with the existing Th1/Th2 paradigm, and reasonable as long as Gata3 is tightly associated with the Th2 phenotype. However, the present data, generated in a productive Chlamydia genital tract infection model that reproduces human pathology including infertility and hydrosalpinx, shows that Chlamydia-specific CD4 T cell clones universally expressed Gata3 and produced IFN-g upon activation, even a CD4 clone with little or no Tbet, violating the basic mutual exclusivity tenets of the Th1/2 paradigm.

### Example 7: CD93(g13Th1) and Gpm6b (g13Th2) are Mutually Exclusive, Subset-Specific Biomarkers

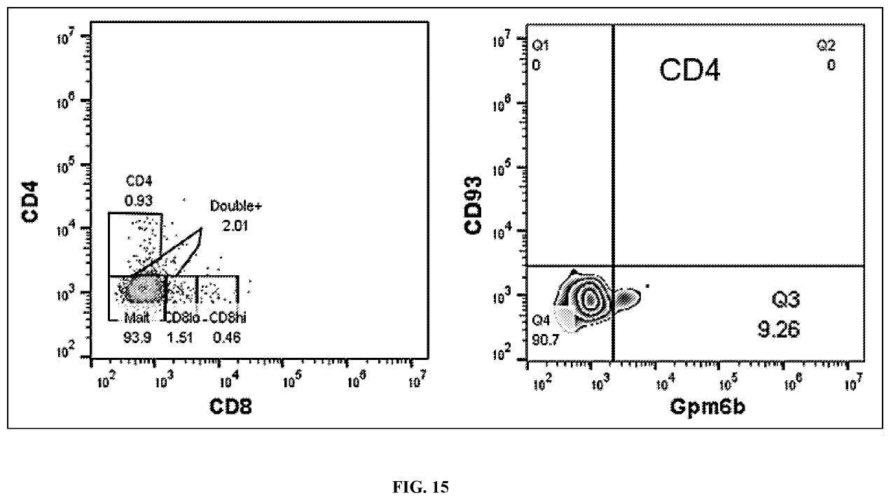

The endometrial mononuclear cells gated on CD3 and CD4 were stained and the expression of CD93 (g13Th1) versus Gpm6b (g13Th2) was analyzed. As shown in FIG. 15, in this one individual, recruited without knowledge of any specific pathological condition, the dominant CD4g13 subset was Gpm6b (g13Th2) positive, representing 9.26% of endometrial CD4 T cells, whereas there were no detectable CD93 positive CD4 T cells. Since, no double positive CD93/Gpm6b cells were observed, it is concluded that CD93 and Gpm6b are mutually exclusive, subset-specific biomarkers.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method for treating an inflammatory or autoimmune disease in a subject, the method comprising administering to the subject an effective amount of a therapeutic agent that increases the population of protective g13Th1 T cells in the subject, thereby treating inflammatory or autoimmune disease in the subject.

2. The method of claim 1, wherein when the therapeutic agent increases the population of protective g13Th1 T cells, an increase in the level of at least one cytokine selected from the group consisting of IL17 and IL22 is detected.

3. The method of claim 1, wherein the population of protective g13Th1 T cells comprise at least one biomarker selected from the group consisting of: Cd93, Large, Cpa3, Pde8a, Pgr, Nm1, Dapk1, Cyp4f39, Noxred1 and Treml2.

4. The method of claim 1, wherein the therapeutic agent decreases the population of pathological g13Th2 T cells thereby decreasing the level of at least one cytokine selected from the group consisting of IL4 and IL5.

5. The method of claim 4, wherein the population of pathological g13Th2 T cells comprises at least one biomarker selected from the group consisting of: Fam213a, Bmp8, Lrrc32, Cyp11a1, tarm1, Chat2, Chil3, Gpm6b, Bace2, Lag3, Acbd7, Ctse, Hsd17b11 and Hrh4.

6. A method for treating an inflammatory or autoimmune disease in a subject, the method comprising administering to the subject an effective amount of a therapeutic agent that decreases the population of pathologic g13Th1 T cells in the subject, thereby treating inflammatory or autoimmune disease in the subject.

7. The method of claim 6, wherein when the therapeutic agent decreases the population of pathologic g13Th1 T cells, a decrease in the level of at least one cytokine selected from the group consisting of IL17 and IL22 is detected.

8. The method of claim 6, wherein the population of pathologic g13Th1 T cells comprise at least one biomarker selected from the group consisting of: Cd93, Large, Cpa3, Pde8a, Pgr, Nm1, Dapk1, Cyp4f39, Noxred1 and Treml2.

9. The method of claim 6, wherein the therapeutic agent increases the population of protective g13Th2 T cells thereby increasing the level of at least one cytokine selected from the group consisting of IL4 and IL5.

10. The method of claim 9, wherein the population of protective g13Th2 T cells comprises at least one biomarker selected from the group consisting of: Fam213a, Bmp8, Lrrc32, Cyp11a1, tarm1, Chat2, Chil3, Gpm6b, Bace2, Lag3, Acbd7, Ctse, Hsd17b11 and Hrh4.

11. A method for treating an inflammatory or autoimmune disease in a subject, the method comprising administering to the subject an effective amount of a therapeutic agent that increases the population of protective g13Th2 T cells in the subject, thereby treating inflammatory or autoimmune disease in the subject.

12. A method for treating an inflammatory or autoimmune disease in a subject, the method comprising administering to the subject an effective amount of a therapeutic agent that decreases the population of pathologic g13Th2 T cells in the subject, thereby treating inflammatory or autoimmune disease in the subject.

13. The method of claim 1, wherein the therapeutic agent is at least one selected from the group consisting of a large molecule, a small molecule, a ligand, an enzyme, a peptidomimetic, an antibody, an aptamer, a vaccine and a combination thereof.

14. A method of assessing the type of immune response in an inflammatory or autoimmune disease in a subject, the method comprising:
a. detecting the level of cytokines IL2, IL13, IL4, IL5, IL17 and IL22 in a sample from the subject;
b. determining the level of gene expression of at least one T cell biomarker selected from the group consisting of, Cd93, Large, Cpa3, Pde8a, Pgr, Nm1, Dapk1, Cyp4f39, Noxred1, Treml2, Fam213a, Bmp8, Lrrc32, Cyp11a1, tarm1, Chat2, Chil3, Gpm6b, Bace2, Lag3, Acbd7, Ctse, Hsd17b11 and Hrh4 in a sample from the subject;
c. comparing the level of the cytokines or the at least one T cell biomarker in the sample from the subject to a baseline level in a control subject not having an inflammatory or autoimmune disease, wherein a higher level of the cytokines or the at least one T cell biomarker in the sample as compared to the level of the cytokines or the at least one T cell biomarker in the control is indicative of a g13Th1 or g13Th2 T cell response; and,
d. wherein when a g13Th1 or g13Th2 T cell response is indicated, treatment of the inflammatory or autoimmune disease is recommended.

15. A method of detecting a pathological T cell response in an inflammatory or autoimmune disease in a subject, the method comprising:
a. determining the presence of a pathological g13Th1 T cell by detecting the level of cytokines IL2, IL13, IL17, IL22 and IFNg in a sample from the subject;
b. determining the level of gene expression of at least one pathological g13Th1 T cell biomarker selected from the group consisting of: Cd93, Large, Cpa3, Pde8a, Pgr, Nm1, Dapk1, Cyp4f39, Noxred1, Treml2;
c. comparing the level of the cytokines or the at least one g13Th1 T cell biomarker in the sample from the subject to a baseline level in a control subject not having an inflammatory or autoimmune disease, wherein a higher level of the cytokines or the at least one g13Th1 T cell biomarker in the sample as compared to the level of the cytokines or the at least one g13Th1 T cell biomarker in the control is indicative of a pathological T cell response; and,
d. wherein when a pathological T cell response is indicated, treatment of the inflammatory or autoimmune disease is recommended.

16. A method of detecting a pathological T cell response in an inflammatory or autoimmune disease in a subject, the method comprising:
a. determining the presence of a pathological g13Th2 T cell by detecting the level of cytokines IL2, IL13, IL4, IL5 and IFNg in a sample from the subject;
b. determining the level of gene expression of at least one g13Th2 T cell biomarker selected from the group consisting of: Fam213a, Bmp8, Lrrc32, Cyp11a1, tarm1, Chat2, Chil3, Gpm6b, Bace2, Lag3, Acbd7, Ctse, Hsd17b11 and Hrh4;
c. comparing the level of the cytokines or the at least one g13Th2 T cell biomarker in the sample from the subject to a baseline level in a control subject not having an inflammatory or autoimmune disease, wherein a higher level of the cytokines or the at least one g13Th2 T cell biomarker in the sample as compared to the level of the cytokines or the at least one g13Th2 T cell biomarker in the control is indicative of a pathological T cell response; and,
d. wherein when a pathological T cell response is indicated, treatment of the inflammatory or autoimmune disease is recommended.

17. A method of detecting a protective T cell response in an inflammatory or autoimmune disease in a subject, the method comprising:
a. determining the presence of a protective g13Th1 T cell by detecting the level of cytokines IL2, IL13, IL17, IL22 and IFNg in a sample from the subject;
b. determining the level of gene expression of at least one g13Th1 T cell biomarker selected from the group consisting of: Cd93, Large, Cpa3, Pde8a, Pgr, Nm1, Dapk1, Cyp4f39, Noxred1 and Treml2;
c. comparing the level of the cytokines or the at least one g13Th1 T cell biomarker in the sample from the subject to a baseline level in a control subject not having an inflammatory or autoimmune disease, wherein a higher level of the cytokines or the at least one g13Th1 T cell biomarker in the sample as compared to the level of the cytokines and/or to the at least one g13Th1 T cell biomarker in the control is indicative of a protective T cell response; and,
d. wherein when a protective T cell response is indicated, treatment of the inflammatory or autoimmune disease is recommended.

18. A method of detecting a protective T cell response in an inflammatory or autoimmune disease in a subject, the method comprising:
a. determining the presence of a protective g13Th2 T cell by detecting the level of cytokines IL2, IL13, IL4, IL5 and IFNg in a sample from the subject;
b. determining the level of gene expression of at least one g13Th2 T cell biomarker selected from the group consisting of: Fam213a, Bmp8, Lrrc32, Cyp11a1, tarm1, Chat2, Chil3, Gpm6b, Bace2, Lag3, Acbd7, Ctse, Hsd17b11 and Hrh4;
c. comparing the level of the cytokines or the at least one g13Th2 T cell biomarker in the sample from the subject to a baseline level in a control subject not having an inflammatory or autoimmune disease, wherein a higher level of the cytokines or the at least one g13Th2 T cell biomarker in the sample as compared to the level of the cytokines and/or to the at least one g13Th2 T cell biomarker in the control is indicative of a protective T cell response; and,
d. wherein when a protective T cell response is indicated, treatment of the inflammatory or autoimmune disease is recommended.

19. The method of claim 14, wherein the sample is selected from the group consisting of blood, endometrial biopsy, stool and synovial fluid.

20. A method for stimulating a protective CD4g13 T cell-mediated immune response to a cell population or a local tissue or organ in a subject in need thereof, the method comprising administering to the subject an effective amount of a therapeutic agent that increases the population of protective CD4g13 T cells and a pharmaceutical acceptable carrier.

21. The method of claim 20, wherein the population of protective CD4g13 T cells consists of at least one selected from the group consisting of protective g13Th1 cells and protective g13Th2 cells.

22. (canceled)

23. (canceled)

24. The method of claim 1, wherein the inflammatory or autoimmune disease is linked to at least one infection selected from the group consisting of bacterial, viral and parasitic.

25. The method or kit of claim 24, wherein the bacterial infection is a Chlamydia bacterium.

26. The method of claim 1, wherein the inflammatory or autoimmune disease is selected from the group consisting of mucosal inflammation, orchitis, epididymis, inflammatory bowel disease, Crohn's disease, ulcerative colitis, multiple sclerosis, rheumatoid arthritis, and psoriasis.

27. The method of claim 1, wherein the subject is a mammal.

28. The method or kit of claim 27, wherein the mammal is a human.

29. A pharmaceutical composition for treating an inflammatory or autoimmune disease in a subject, the pharmaceutical composition comprising a therapeutic agent that increases the population of protective g13Th1 T cells and a pharmaceutical acceptable carrier.

30. The pharmaceutical composition of claim 29, wherein the population of protective g13Th1 T cells comprises at least one biomarker selected from the group consisting of: Cd93, Large, Cpa3, Pde8a, Pgr, Nm1, Dapk1, Cyp4f39, Noxred1 and Treml2.

31. A pharmaceutical composition for treating an inflammatory or autoimmune disease in a subject, the pharmaceutical composition comprising a therapeutic agent that increases the population of protective g13Th2 T cells and a pharmaceutical acceptable carrier.

32. The pharmaceutical composition of claim 31, wherein the population of protective g13Th2 T cells comprises at least one biomarker selected from the group consisting of: Fam213a, Bmp8, Lrrc32, Cyp11a1, tarm1, Chat2, Chil3, Gpm6b, Bace2, Lag3, Acbd7, Ctse, Hsd17b11 and Hrh4.

33. A pharmaceutical composition for treating an inflammatory or autoimmune disease in a subject, the pharmaceutical composition comprising a therapeutic agent that decreases the population of pathological g13Th1 cells and a pharmaceutical acceptable carrier.

34. The pharmaceutical composition of claim 33, wherein the population of pathologic g13Th1 T cells comprises at least one biomarker selected from the group consisting of: Cd93, Large, Cpa3, Pde8a, Pgr, Nm1, Dapk1, Cyp4f39, Noxred1 and Treml2.

35. A pharmaceutical composition for treating an inflammatory or autoimmune disease in a subject, the pharmaceutical composition comprising a therapeutic agent that decreases the population of pathological g13Th2 cells and a pharmaceutical acceptable carrier.

36. The pharmaceutical composition of claim 35, wherein the population of pathological g13Th2 T cells comprises at least one biomarker selected from the group consisting of: Fam213a, Bmp8, Lrrc32, Cyp11a1, tarm1, Chat2, Chil3, Gpm6b, Bace2, Lag3, Acbd7, Ctse, Hsd17b11 and Hrh4.

37. (canceled)

38. (canceled)

39. The pharmaceutical composition of claim 29, wherein the therapeutic agent is at least one selected from the group consisting of a large molecule, a small molecule, a ligand, an enzyme, a peptidomimetic, an antibody, an aptamer, a vaccine and a combination thereof.

40. (canceled)

41. (canceled)

42. (canceled)

43. (canceled)
